(19) United States
(12) Patent Application Publication
(10) Pub. No.: US 2025/0255799 A1
(43) Pub. Date: Aug. 14, 2025

(54) METHOD FOR PRODUCING HYDROGEL

(71) Applicant: TEOXANE SA, GENEVE (CH)

(72) Inventors: Elodie CLERC, FILLIERE (FR); Jimmy FAIVRE, VALSERHONE (FR)

(21) Appl. No.: 18/857,059

(22) PCT Filed: Apr. 14, 2023
(86) PCT No.: PCT/EP2023/059835

§ 371 Date: Oct. 15, 2024

(30) Foreign Application Priority Data
Apr. 15, 2022 (FR) FR2203536
Apr. 15, 2022 (FR) FR2203541
Aug. 16, 2022 (FR) FR2208327

(51) Int. Cl. A61K 8/73; A61K 8/04; A61Q 19/08; C08J 3/075; C08J 3/24

(52) U.S. Cl. CPC A61K 8/735; A61K 8/042; A61Q 19/08; C08J 3/075; C08J 3/24; A61K 2800/91; C08J 2305/08

(57) ABSTRACT

The present invention relates to a method for producing a hydrogel comprising a crosslinked polysaccharide, in particular, a method for producing an injectable hydrogel comprising crosslinked hyaluronic acid. The hydrogel has mechanical properties suitable for filling soft tissues. The present invention also relates to a hydrogel, preferably injectable, that can be obtained by the method and a composition containing the hydrogel.

### FIELD OF THE INVENTION

The present invention relates to a method for producing a hydrogel comprising a crosslinked polysaccharide, in particular, a method for producing an injectable hydrogel comprising crosslinked hyaluronic acid. The hydrogel has mechanical properties suitable for filling soft tissues. The present invention also relates to a hydrogel, preferably injectable, that can be obtained by the method and a composition containing the hydrogel.

### TECHNICAL BACKGROUND

Polysaccharides, such as glycosaminoglycans, are widely used in the medical and aesthetic fields, in particular for filling soft tissues.

In particular, the majority of commercialised products for aesthetic applications are hyaluronic acid-based.

In order to improve the quality of the skin, the gels prepared from non-modified hyaluronic acid are of interest because they have the advantage of being perfectly biocompatible.

However, after implantation in vivo, they degrade very rapidly. Thus, the effects of such gels are only of short duration.

In order to increase the sustainability in vivo of hyaluronic acid-based gels and their resistance to degradation, the hyaluronic acid is usually modified by crosslinking.

Crosslinked hyaluronic acid-based gels can be obtained by various preparation methods.

In particular, polysaccharides, including hyaluronic acid, can be modified by means of crosslinking agents, such as epoxy agents, aldehydes such as glutaraldehyde, divinyl sulfone (DVS) or polyamines. Currently, the most commonly used crosslinking agent is 1,4-butanediol diglycidyl ether (BDDE). The polysaccharides can be modified under various conditions of pH, duration, pressure and temperature.

For product safety and performance reasons, it is sought to provide compositions which are sustainable in vivo comprising a biocompatible polysaccharide that is the most natural and therefore the least modified, in particular the least crosslinked.

In order to reduce the quantity of crosslinking agent necessary for obtaining a polysaccharide-based gel having mechanical properties suitable for its use, various modifications of the crosslinking process parameters have already been proposed, such as adding various salts, increasing the concentration of hyaluronic acid and/or sodium hydroxide in the crosslinking medium or else, adjusting the duration and the temperature of the crosslinking reaction (WO2014/064633; WO2016/096920; WO2017/016917; Sukwha Kim et al., Carbohydrate Polymers, 2018, 202, 545-553).

However, below a certain threshold, the gels prepared no longer have suitable mechanical properties. In particular, crosslinked hyaluronic acid gels of 1,4-butanediol diglycidyl ether (BDDE) with a degree of modification of approximately 1% are not very cohesive and/or do not withstand the heat-sterilisation conditions generally applied to this type of product (autoclave sterilisation). These products which are unstable during sterilisation, ultimately have a predominantly viscous component, which is not desirable for soft tissue filling applications.

Thus, there remains a need to provide a method for preparing a polysaccharide-based gel, in particular based on hyaluronic acid, which can further reduce the quantities of conventional crosslinking agent used, while enabling the prepared gel to have mechanical properties suitable for filling soft tissues.

### BRIEF DESCRIPTION OF THE INVENTION

An object of the invention is a method for preparing a polysaccharide-based hydrogel, comprising the following steps:
a) providing at least one polysaccharide;
b) providing at least one crosslinking agent and/or at least one functionalisation agent, the functionalisation agent enabling crosslinking of the polysaccharide by sol-gel reaction;
c) preparing a reaction medium comprising a solvent, the one or more polysaccharides and the one or more crosslinking agents and/or functionalisation agents;
d) crosslinking the polysaccharide:
   d1) by reacting the polysaccharide with the one or more crosslinking agents; or
   d2) by sol-gel reaction of the functionalised polysaccharide, the functionalised polysaccharide being obtained by reaction of the polysaccharide with the one or more functionalisation agents;
   wherein the crosslinking of the polysaccharide according to d1) or d2) is carried out under conditions not allowing sublimation of water, at a pressure P and at a temperature T greater than the temperature of the eutectic point of the reaction medium as measured at pressure P and less than the temperature of the freezing point of the reaction medium as measured at pressure P.

Advantageously, the crosslinking agent comprises at least two functional groups Z, identical or different, chosen from the isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy or carbodiimide groups, and an acid anhydride residue.

Advantageously, the functionalisation agent is a molecule Chem. II having the following formula:

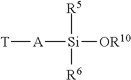

wherein:
T represents an isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy or carbodiimide group, or an acid anhydride residue;
A represents a chemical bond or a spacer group;
R5 and R6, identical or different, represent a hydrogen atom; a halogen atom; an —OR4 group with R4 representing a hydrogen atom, an aryl group or an aliphatic hydrocarbon group having 1 to 6 carbon atoms; an aryl; or an aliphatic hydrocarbon group having 1 to 6 carbon atoms optionally substituted by one or more groups chosen from a halogen atom, an aryl and a hydroxyl group;
R10 represents a hydrogen atom, an aryl group or an aliphatic hydrocarbon group having 1 to 6 carbon atoms.

The functional groups Z are advantageously identical and represent an epoxide or vinyl group, more preferably epoxide.

The crosslinking agent is advantageously selected from the group consisting of 1,4-butanediol diglycidyl ether (BDDE), 1,2,7,8-diepoxy-octane, poly(ethylene glycol) diglycidyl ether (PEGDGE), 1,2-bis(2,3-epoxypropoxy)ethane (EGDGE), 1,3-bis(3-glycidyloxypropyl)tetramethyldisiloxane, poly(dimethylsiloxane) terminated at each end by a diglycidyl ether (CAS number: 130167-23-6), hydroxyapatite beads modified to carry epoxy groups and the mixtures thereof.

The quantity of crosslinking agent advantageously varies from 0.001 to 0.15 mole per 1 mole of polysaccharide repetition unit, preferably from 0.001 to 0.08 mole per 1 mole of polysaccharide repetition unit, yet more preferably 0.001 to 0.05 mole per 1 mole of polysaccharide repetition unit.

The crosslinking of the polysaccharide according to d1) or d2) is advantageously carried out for a duration of at least 1 hour, preferably at least 3 hours, preferably at least 72 hours, preferably at most 27 weeks, under conditions not allowing sublimation of water at temperature T.

The crosslinking of the polysaccharide according to d1) or d2) is advantageously carried out for a duration ranging from 2 to 25 weeks, preferably ranging from 2 to 20 weeks or 2 to 17 weeks, yet more preferably from 3 to 8 weeks or from 4 to 7 weeks, under conditions not allowing sublimation of water at temperature T.

The pressure P is advantageously less than or equal to atmospheric pressure, advantageously between 0.7·10⁵ Pa and 0.9·10⁵ Pa or equal to atmospheric pressure.

Step d) is advantageously carried out in a hermetically sealed container which can be flexible or rigid, advantageously flexible.

During step d) the hermetic container is advantageously placed at a temperature ranging from −35° C. to −10° C., at atmospheric pressure, preferably at a temperature of approximately −20° C., at atmospheric pressure.

Another object of the invention is a hydrogel which may be obtained by the method according to the invention.

Another object of the invention is a cosmetic or pharmaceutical composition comprising a hydrogel according to the invention.

Another object of the invention is the hydrogel according to the invention or the composition according to the invention for use thereof in the filling and/or replacement of tissues.

Another object of the invention is the cosmetic use of a hydrogel according to the invention or a composition according to the invention for preventing and/or treating the change in the viscoelastic or biomechanical properties of the skin; for filling volume defects of the skin, in particular for filling wrinkles, fine lines and scars; for attenuating the nasolabial folds and bitterness folds; for increasing the volume of the cheekbones, the chin or lips; for restoring the volumes of the face, in particular the cheeks, temples, the oval of the face, and around the eye; for reducing the appearance of wrinkles and fine lines; or to stimulate, regenerate, hydrate, firm or restore the radiance of the skin, in particular by mesotherapy.

### FIGURES

FIG. 1 shows a photograph after thawing of the gel of prototype A of example 2.

FIG. 2 shows a photograph after thawing of the gel of prototype B of example 2.

### DEFINITIONS

“Atmospheric pressure” is the pressure that is exerted by the air which constitutes the atmosphere on any surface in contact with it. It varies as a function of altitude.

At an altitude of 0 m, the average air pressure is 101,325 Pa.

The term “gel” designates a polymer network which is dilated over its entire volume by a fluid. This means that a gel is formed of two media, one “solid” and the other “liquid”, dispersed in one another. The medium referred to as “solid” consists of long-molecule polymers connected to one another by weak bonds (for example hydrogen bonds) or covalent bonds (crosslinking). The liquid medium consists of a solvent. A gel generally corresponds to a product which has a phase angle δ less than or equal to 45° at 1 Hz for deformation of 0.1% or a pressure of 1 Pa, advantageously a phase angle δ ranging from 2° to 45° or from 20° to 45°.

The term “hydrogel” designates a gel as defined above, wherein the solvent constituting the liquid medium is mostly water (for example at least 90%, in particular at least 95%, especially at least 99%, by weight of the liquid medium).

Preferably, the liquid medium comprises, in particular consists of, a buffer solution, advantageously enabling a pH of the liquid medium between 6.8 and 7.8, in particular a phosphate-buffered saline.

The term “injectable gel” designates a gel that can flow and be injected manually by means of a syringe equipped with a needle of diameter ranging from 0.1 to 0.5 mm, for example a 30 G, 27 G, 26 G, 25 G hypodermic needle. Preferably, an “injectable gel” is a gel having a mean extrusion force less than or equal to 25 N, preferably ranging from 5 to 25 N, more preferably ranging from 8 to 15 N, during a measurement with a dynamometer, at a fixed speed of approximately 12.5 mm/min, in syringes of external diameter greater than or equal to 6.3 mm, with a needle of external diameter less than or equal to 0.4 mm (27G) and length ½″, at ambient temperature.

The property “stretchiness” of a product designates its ability to be stretched between two surfaces to which it has adhered. The stretchiness property can be determined using a texturometer, a sensory analysis performed by a panel, or else rheological and mechanical measurements including, in particular, measurement of the phase angle (δ), G′ and G″, or tensile tests. In particular, this property can be measured as described by P. Micheels et al. (Micheels et al., Comparison of two swiss-designed hyaluronic acid gels: six-month clinical follow-up, Journal of Drug in Dermatology, 2017, 16:154-161, “Resistance to stretching”) or by carrying out a Tack test and by measuring the length of the threads under traction.

The “stickiness” property of a product designates its ability to adhere to a surface. It can be determined qualitatively using a sensory analysis performed by a panel or else by moving a bolus on a surface. It can also be determined quantitatively by measuring the adhesion force to a surface by tensile testing machine or mechanical analysis.

The term “polysaccharide” designates a polymer composed of monosaccharides (preferably D-enantiomers) joined together by glycosidic bonds.

The term “monosaccharide”, also referred to as an “ose”, designates a non-modified or modified monosaccharide.

A non-modified “monosaccharide” designates a compound of formula H—(CHOH)ₓ—CO—(CHOH)_{y}—H with x and y representing, independently of one another, an integer ranging from 0 to 5 under the condition that 2≤x+y≤5, the monosaccharide can be in a linear form represented by the above-mentioned formula or can be in a cyclic form by reaction of the CO function (aldehyde or ketone) with one of the OH groups in order to form a hemiacetal or hemiketal. Preferably, the monosaccharide is in cyclic form. There are two types of ose: aldoses, which carry an aldehyde function (when x or y equals 0) and ketoses which carry a ketone function (when neither x, nor y equals 0). Monosaccharides are classified by number of carbons. For example, monosaccharides with 6 carbons (x+y=5) are hexoses of formula C₆H₁₂O₆ and can be allose, altrose, glucose, mannose, gulose, idose, galactose or talose. Monosaccharides with 5 carbons (x+y=4) are pentoses of formula C₅H₁₀O₅ and can be ribose, arabinose, xylose, or lyxose. The monosaccharide is preferably a hexose, in other words x+y=5.

A monosaccharide further comprises x+y asymmetric carbons and therefore 2^{(x+y-1)} enantiomer pairs. Each enantiomer pair is designated by a different name and the enantiomers of a same pair are respectively qualified as D and L enantiomers.

A “modified monosaccharide” designates a non-modified monosaccharide as defined above for which, for example:
one or more OH functional groups have been replaced by another functional group, for example:
(i) an OR group with R representing a (C₁-C₆)alkyl group such as methyl or ethyl; a hydroxy-(C₁-C₆)alkyl groups such as hydroxyethyl (—CH₂CH₂OH) or hydroxypropyl (—CH₂—CH(OH)—CH₃); a carboxy-(C₁-C₆)alkyl group such as carboxymethyl (—CH₂COOH); or CO—(C₁-C₆)alkyl groups such as acetyl; and/or
(ii) an NR′R″ group with R′ and R″ representing, independently of one another, H, (C₁-C₆)alkyl or CO—(C₁-C₆)alkyl such as acetyl; and/or
(iii) an OSO₃H group; and/or
   the one or more CH₂OH end functions have been replaced by a COOH or CHO group;
   a —CH(OH)—CH(OH)— bond is oxidised to give two —CHO (aldehyde) end groups in place of this bond; and/or
   a CH₂OH end function has been condensed with an OH functional group in order to form an —O—CH₂— chain.

The expression “repetition unit” of a polysaccharide designates a structural unit consisting of one or more (generally 1 or 2) monosaccharides, the repetition of which produces the complete polysaccharide chain.

Some or all of the monosaccharides can be in a modified form.

The monosaccharides, when they are modified, can be in different modified forms.

The term “physiologically acceptable” designates that which is generally safe, non-toxic and neither biologically nor otherwise undesirable and which is acceptable for cosmetic use (in other words non-therapeutic use) or for human or veterinary therapeutic use, in particular for use by injection into the human or animal body or for a topical application on the skin.

The “salts” used in the context of the present invention are preferably physiologically acceptable salts. The term “physiologically acceptable salts” designates, in particular:
(1) the pharmacologically acceptable acid addition salts formed with pharmaceutically acceptable inorganic acids such as hydrochloric acid, hydrobromic acid, the sulfuric acid, nitric acid, phosphoric acid and similar; or formed with pharmaceutically acceptable organic acids, such as formic acid, acetic acid, benzenesulfonic acid, benzoic acid, camphorsulfonic acid, citric acid, ethane-sulfonic acid, the fumaric acid, glucoheptonic acid, gluconic acid, glutamic acid, glycolic acid, hydroxynaphtoic acid, 2-hydroxyethanesulfonic acid, lactic acid, maleic acid, malic acid, mandelic acid, methanesulfonic acid, muconic acid, 2-naphtalenesulfonic acid, propionic acid, salicylic acid, succinic acid, dibenzoyl-L-tartric acid, tartric acid p-toluenesulfonic acid trimethylacetic acid, trifluoroacetic acid and similar, and
(2) pharmacologically acceptable base addition salts formed when an acid proton present in the parent compound is either replaced by a metal ion, for example an alkali metal ion (e.g. Na, K), alkaline earth metal ion (e.g., Ca, Mg), a zinc ion, a silver ion or an aluminium ion; or coordinated with a pharmaceutically acceptable organic base such as diethanolamine, ethanolamine, N-methylglucamine, triethanolamine, tromethamine and similar; or with a pharmaceutically acceptable inorganic base, such as aluminium hydroxide, calcium hydroxide, potassium hydroxide, sodium carbonate, sodium hydroxide and similar.

The “degree of modification” (MOD), expressed in %, of a polysaccharide, such as hyaluronic acid, corresponds to the molar quantity of modifying agent, such as the quantity of crosslinking agent and/or the functionalisation agent bonded to the polysaccharide, by one or more of its ends, expressed per 100 moles of polysaccharide repetition units. It can be determined by the methods known to a person skilled in the art, such as nuclear magnetic resonance spectroscopy (NMR).

The “molar crosslinking ratio” (TR), expressed in %, designates the molar ratio of the quantity of crosslinking agent to the quantity of polysaccharide repetition unit introduced into the crosslinking reaction medium, expressed per 100 moles of polysaccharide repetition units in the crosslinking medium.

The “molar functionalisation ratio”, expressed in %, designates the molar ratio of the quantity of functionalisation agent to the quantity of polysaccharide repetition unit introduced into the crosslinking reaction medium, expressed per 100 moles of polysaccharide repetition units in the functionalisation medium.

The expression “therapeutic active ingredient” designates a substance for curing, relieving the symptoms of and/or preventing a disease; a substance having curative or preventative properties with respect to human or animal diseases, as well as any substance which can be used in humans or in animals or which can be administered to them, with a view to establishing a medical diagnosis or restoring, correcting or modifying their physiological functions by exerting a pharmacological, immunological or metabolic action.

The expression “cosmetic active ingredient” designates any non-therapeutic substance, in particular intended to be placed in contact with various superficial parts of the human body, such as the epidermis, the hair and capillary systems, nails, lips, chest and teeth, with a view, exclusively or mainly, to cleaning, protecting, or perfuming them, maintaining them in good condition, modifying their appearance or odour.

The term “approximately” designates that the value concerned can be less than or greater than the indicated value by 10%, notably by 5%, in particular by 1%.

An “aqueous reaction medium” designates a reaction medium for which the solvent is mostly water (for example at least 90%, in particular at least 95%, in particular at least 99% by total weight of the solvent) or is water.

The expression “spacer group” designates a fragment comprising at least one atom intended to link together two chemical groups within a same molecule. The spacer group preferably contains at least one carbon atom.

The term “halogen” designates an atom of fluorine, chlorine, bromine or iodine.

An “epoxide” group is an ethylene oxide residue linked to the remainder of the molecule by one of its carbon atoms.

An “N-succinimidyloxycarbonyl” group is a group of formula Chem. GR1 below:

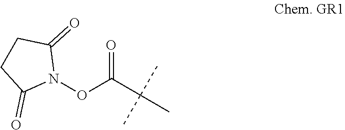

An “N-sulfosuccinimidyloxycarbonyl” group is a group of formula Chem. GR2 below:

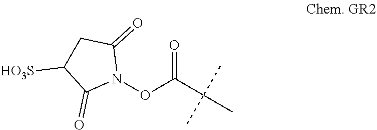

A “halogenocarbonyl” group is a group of formula —CO-Hal with Hal representing a halogen, such as Cl or Br.

A “carbodiimide” group is a group comprising an —N═C═N— unit, and more particularly a group of formula —N═C═N—R^{a} with R^{a} representing an aliphatic hydrocarbon group having 1 to 20 carbon atoms, preferably a (C1-C6)alkyl group, for which one or more carbon atoms are optionally replaced by a heteroatom chosen from O, S and N, in particular N.

An “acid anhydride residue” is a group comprising a —C(O)—O—C(O)—) unit, and more particularly a monovalent cyclic group comprising the —C(O)—O—C(O)— unit, such as a saturated monovalent hydrocarbon monocyclic group comprising 5 to 10, in particular 5 or 6, carbon atoms, of which three successive carbon atoms are replaced by C(O)—O—C(O) and optionally one or more of which, in particular one, additional carbon atoms, preferably not consecutive with the three carbon atoms substituted by CO—O—CO, are each replaced by a heteroatom such as N, O or S, in particular N. The acid anhydride residue may respond in particular to the following formula Chem. GR3:

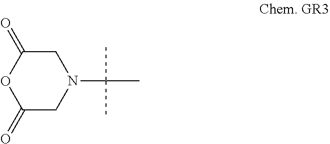

The acid anhydride residue can also be chosen from a maleic anhydride residue or a succinic anhydride residue.

The expression “aliphatic hydrocarbon chain” or “aliphatic hydrocarbon group” designates a linear, branched and or cyclic, saturated or unsaturated, but not aromatic, hydrocarbon group, advantageously comprising 1 to 50, in particular 1 to 20, for example 1 to 12 or 1 to 6 carbon atoms. It involves, in particular, alkyl groups

The expression “branched aliphatic hydrocarbon chain” specifically designates a main aliphatic hydrocarbon chain comprising at least one secondary aliphatic hydrocarbon chain.

The expression “star-shaped aliphatic hydrocarbon chain” designates a branched aliphatic hydrocarbon chain comprising a plurality of secondary aliphatic hydrocarbon chains all starting from a single branching point.

The expression “alkyl with C1-Cx” or “(C1-Cx)alkyl” or else “alkyl having 1 to x carbon atoms” designates a saturated, linear or branched, monovalent hydrocarbon group, having 1 to x carbon atoms, with x an integer, for example a methyl, ethyl, isopropyl, tertio-butyl, n-pentyl, cyclopropyl or cyclohexyl group, etc.

The expression “(C1-Cx)alkylene” designates a saturated, linear or branched, divalent hydrocarbon group comprising 1 to x carbon atoms, with x an integer, for example a methane-1,1-diyl, ethane-1,1-diyl, ethane-1,2-diyl, propane-1,3-diyl, butane-1,4-diyl, butane-1,3-diyl, butane-1,2-diyl, pentane-1,5-diyl, hexane-1,6-diyl, hexane-1,5-diyl, heptane-1,7-diyl, octane-1,8-diyl, nonane-1,9-diyl or decane-1,10-diyl group, etc. It involves, in particular, a methane-1,1-diyl or propane-1,3-diyl group.

The expression “hydroxy-(C1-Cx)alkyl” designates a (C1-Cx)alkyl group such as defined above substituted by a hydroxyl (OH) group, for example a hydroxyethyl (—CH₂CH₂OH) or a hydroxypropyl (for example —CH₂—CH(OH)—CH₃).

The expression “carboxy-(C1-Cx)alkyl” designates a (C1-Cx)alkyl group such as defined above, substituted by a carboxyl (COOH) group, for example a carboxymethyl (—CH₂COOH) group.

The expression “aryl” designates a monovalent aromatic hydrocarbon group, preferably having 6 to 10 carbon atoms, comprising one or more cycles, for example a phenyl or naphtyl group.

The expression “arylene” designates a divalent aromatic hydrocarbon group, preferably having 6 to 10 carbon atoms, comprising one or more cycles, such as a phenylene group.

The expression “aryl-(C1-Cx)alkyl” designates an aryl group such as defined above, linked to the rest of the molecule by means of a (C1-Cx)alkyl chain as defined above, with x an integer, for example the benzyl or phenylethyl group.

The expression “polyvalent group” designates a group which can form a plurality of covalent bonds with other groups of a same compound or of two different compounds.

The bonds to the other groups can be formed from the same atom of the polyvalent group or from different atoms of the polyvalent group, and preferably from different atoms of the polyvalent group. In particular, the polyvalent group is a divalent group and can therefore form two covalent bonds with two other groups of the same compound or of two different compounds. The number of covalent bonds that can be formed designates the “valence” of the polyvalent group.

The expression “partially concomitant” as used in expressions of the type “steps b) and c) are partially concomitant” means that the two steps are carried out, in part, at the same time, under the same reaction conditions, but that at least one of the two steps is initiated or terminated under different reaction conditions from the common reaction conditions.

The expressions “between X and XX” or “from X to XX”, X and XX representing a numerical value, are synonyms used in equivalent manner. In all cases, the limit values are included in the envisaged range.

### DETAILED DESCRIPTION OF THE INVENTION

The inventors have developed a method for preparing of a polysaccharide-based hydrogel responding to the expressed needs.

The proposed preparation method enables a crosslinked polysaccharide-based hydrogel to be obtained, which is ideally weakly crosslinked, in particular crosslinked hyaluronic acid, advantageously weakly crosslinked, that has mechanical properties suitable for use in filling soft tissues.

The proposed method comprises the following steps:
a) providing at least one polysaccharide;
b) providing at least one crosslinking agent and/or at least one functionalisation agent, the functionalisation agent enabling crosslinking of the polysaccharide by sol-gel reaction;
c) preparing a reaction medium comprising a solvent, the one or more polysaccharides and the one or more crosslinking agents and/or functionalisation agents;
d) crosslinking the polysaccharide:
   d1) by reacting the polysaccharide with the one or more crosslinking agents; or
   d2) by sol-gel reaction of the functionalised polysaccharide, the functionalised polysaccharide being obtained by reaction of the polysaccharide with the one or more functionalisation agents;
wherein the crosslinking of the polysaccharide according to d1) or d2) is carried out under conditions not allowing sublimation of water, at a pressure P and at a temperature T greater than the temperature of the eutectic point of the reaction medium as measured at pressure P and less than the temperature of the freezing point of the reaction medium as measured at pressure P.

In other words, at temperature T, the reaction medium is frozen. The crosslinking of the polysaccharide in the frozen state can preserve the polysaccharide chains, including in an acid or base medium, thus enabling an extension of the reaction time. This extension of the reaction time makes it possible to reduce the quantity of crosslinking and/or modifying agent used, and thus to obtain products that are always more natural.

Furthermore, carrying out the crosslinking step under conditions which do not allow the sublimation of water surprisingly makes it possible to avoid the formation of a gel comprising inhomogeneous, white and brittle crosslinked zones, with a low swelling capacity, which can appear when the polysaccharide is held in a frozen state for a prolonged duration of at least 1 hour, preferably at least 3 hours, preferably at least 72 hours. For example, said duration may range from 2 to 27 weeks, preferably from 2 to 20 weeks, more preferably from 2 to 17 weeks, yet more preferably from 3 to 8 weeks, yet more preferably from 4 to 7 weeks. Gels having such inhomogeneous, white and brittle zones are not suitable for filling soft tissues.

The hydrogels obtained by the method of the present invention are biocompatible and advantageously have a stickiness property enabling the gel to adhere to the tissues in order not to migrate.

Another object of the present invention is a hydrogel which may be obtained by the method according to the invention.

Another object of the present invention is a composition comprising a hydrogel according to the invention, as well as the therapeutic, cosmetic or aesthetic applications of the hydrogels or compositions according to the invention.

### Method

The steps of the method of the present invention can be as described below.

### Providing at Least One Polysaccharide (Step a))

Step a) of the method according to the invention consists of providing at least one polysaccharide. The polysaccharide can be in the form of a salt.

The polysaccharide can be any polymer composed of monosaccharides joined together by glycosidic bonds.

Preferably, the polysaccharide is chosen from pectin and pectic substances; chitosan; chitin; cellulose and its derivatives; agarose; glycosaminoglycans such as hyaluronic acid, heparosan, dermatan sulphate, keratan sulphate, chondroitin and chondroitin sulphate; and the mixtures thereof.

The “pectic substances”, including “pectin”, are polysaccharides composed by a D-galacturonic acid skeleton in acid form, possibly esterified by methanol, and L-rhamnose capable of forming branches with other oses.

“Chitosan” and “chitin” are both polysaccharides composed of D-glucosamine repetition units bonded together in β-(1,4) a part of which is N-acetylated. More particularly, chitosan as a degree of acetylation less than 50%, whereas chitin has more particularly a degree of acetylation greater than 50%.

“Cellulose” is a polysaccharide composed of a linear chain of D-glucose molecules.

The “cellulose derivatives” comprise methylcellulose, ethylcellulose, ethylmethylcellulose, hydroxypropylmethylcellulose (HPMC), hydroxyethylcellulose (HEC), hydroxypropylcellulose (HPC) and carboxymethylcellulose (CMC).

“Agarose” is a polysaccharide comprising, as repetition unit, a disaccharide of D-galactose and of 3,6-anhydro-L-galactopyranose.

“Glycosaminoglycans” are linear polysaccharides composed of repetition units of disaccharides, said disaccharides containing a hexosamine (glucosamine (GlcN) or galactosamine (GalN)) and another ose (glucuronic acid (GlcA), iduronic acid (IdoA) or galactose (Gal)). The hexosamine and the other ose can optionally be sulphated and/or acetylated. The glycosaminoglycan can be, in particular, hyaluronic acid, heparosan, dermatan sulphate, keratan sulphate, chondroitin or chondroitin sulphate.

“Hyaluronic acid” is a glycosaminoglycan for which the repetition unit is a disaccharide composed of D-glucuronic acid and N-acetyl-D-glucosamine, bonded together by alternating glycosidic bonds, β-(1,4) and β-(1,3). When the hyaluronic acid is in the form of a salt, reference is also made to a “hyaluronate” or “hyaluronan”. In the context of the present invention, the hyaluronic acid can have a weight average molar mass between 0.5 and 10 MDa, preferably between 0.5 and 5 MDa, preferably between 0.5 and 4 MDa, preferably between 0.5 and 3 MDa, preferably between 1 and 5 MDa, preferably between 1 and 4 MDa, preferably between 1 and 3 MDa. The hyaluronic acid can be in the form of a salt, in particular in the form of a physiologically acceptable salt, such as the sodium salt, potassium salt, zinc salt, calcium salt, magnesium salt, silver salt, and the mixtures thereof. More particularly, the hyaluronic acid is in acid form or in the form of a sodium salt (NaHA).

“Heparosan” is a glycosaminoglycan for which the repetition unit is a disaccharide composed of glucuronic acid (GlcA) bonded by an α-(1,4) bond to an N-acetyl glucosamine (GlcNAc). Each disaccharide repetition unit is linked to the next by a β-(1,4) bond.

“Chondroitin sulfate” is a glycosaminoglycan for which the repetition unit is a disaccharide composed of glucuronic acid bonded in β(1,3) to sulphated N-acetyl galactosamine, in other words it comprises at least one sulphate substituent. Each disaccharide repetition unit is linked to the next by a β-(1,4) bond.

“Dermatan sulphate” is a glycosaminoglycan for which the repetition unit is a sulphated disaccharide, in other words comprising at least one sulphate substituent, L-iduronic acid and de N-acetyl-galactosamine-bonded by α(1-3) bonds. Advantageously, the disaccharide is sulphated in position C-4 of the N-acetyl-galactosamine, in position C-6 of the N-acetyl-galactosamine, in position C-2 of the L-iduronic acid, or at a combination of these positions. Each disaccharide repetition unit is linked to the next by a β-(1,4) bond.

“Keratan sulphate” is a glycosaminoglycan for which the repetition unit is a sulphated disaccharide, in other words comprising at least one sulphate substituent, composed of D-galactose and N-acetylglucosamine bonded by alternating bonds, β(1-4) and β(1-3).

The polysaccharide can be in the form of a salt, in particular in the form of a physiologically acceptable salt such as the sodium salt, potassium salt, zinc salt, calcium salt, magnesium salt, silver salt and the mixtures thereof, more particularly a sodium or potassium salt.

Advantageously, the polysaccharide is a glycosaminoglycan or a salt thereof, preferably hyaluronic acid or a salt thereof, more preferably hyaluronic acid or one of its physiologically acceptable salts, such as the sodium salt, potassium salt, zinc salt, silver salt, and the mixtures thereof, still more preferably the hyaluronic acid or its sodium salt.

The polysaccharide generally has a weight average molar mass ranging from 0.03 to 10 MDa.

Preferably, if the polysaccharide is hyaluronic acid, it has a weight average molar mass (Mw) ranging from 0.5 to 10 MDa, preferably from 0.5 to 5 MDa, preferably from 0.5 to 4 MDa, preferably from 0.5 to 3 MDa, preferably from 1 to 5 MDa,, preferably from 1 to 4 MDa, preferably from 1 to 3 MDa.

The polysaccharide can be provided in totally or partially hydrated form, or in dry form, such as in the form of a powder or fibres.

In certain embodiments, in step a), the polysaccharide is provided in dry form, such as in the form of a powder or fibres.

When the polysaccharide is provided in hydrated form, it is in the form of a non-crosslinked gel or a solution. In particular, when the polysaccharide is in hydrated form, it is a non-crosslinked aqueous gel or an aqueous solution. More particularly, the polysaccharide is mixed with water, optionally with added phosphate buffer or supplemented phosphate buffer, in other words possibly comprising additional components as described in the section “optional steps”.

### Providing at Least One Crosslinking Agent and/or at Least One Functionalisation Agent (Step b))

Step b) of the method according to the invention consists of providing at least one crosslinking agent and/or at least one functionalisation agent, the functionalisation agent enabling crosslinking of the polysaccharide by sol-gel.

The crosslinking agent and the functionalisation agent are as described above.

### Crosslinking Agent

The “agent for crosslinking”, again commonly and interchangeably designated “crosslinking agent”, is typically a compound comprising at least two functional groups which are able to covalently bond with functional groups present on the polysaccharide, such as OH, CHO, NH₂ or COOH groups carried by the polysaccharide, and thus to induce bonds between the polysaccharide chains (crosslinking) and/or bonds on a same polysaccharide chain.

The crosslinking agent can be in the form of a salt, in particular in the form of a physiologically acceptable salt.

The crosslinking agent used in the context of the present invention comprises at least two, preferably 2 to 8, in particular 2, functional groups (designated “Z groups”) preferably independently chosen from isocyanate (—N═C═O), amino (—NH₂), epoxide, carboxyl (—COOH), N-succinimidyloxycarbonyl, N-sulfosuccinimidyloxycarbonyl, halogenocarbonyl, isothiocyanate (—N═C═S), vinyl (—CH═CH₂), formyl (—CH═O), hydroxyl (—OH), sulfhydryl (—SH), hydrazino (—NH—NH₂), acylhydrazino (—CO—NH—NH₂), aminoxy (—O—NH₂), and carbodiimide groups, and an acid anhydride residue. The functional groups are preferably identical.

The isocyanate can react with an OH or NH₂ group of the polysaccharide to form a carbamate or urea function. The amino group can react with a COOH group of the polysaccharide to form an amide function. The epoxide group can react with an OH or COOH group of the polysaccharide to form an ether or ester function. The carboxyl group can react with an OH or NH₂ group of the polysaccharide to form an ester or amide function. The N-succinimidyloxycarbonyl and N-sulfosuccinimidyloxycarbonyl groups can react with an OH or NH₂ group of the polysaccharide to form an ester or amide function.

The halogenocarbonyl can react with an OH or NH₂ group of the polysaccharide to form an ester or amide function. The isothiocyanate group can react with an OH or NH₂ group of the polysaccharide to form a thiocarbamate or thiourea function. The vinyl group can react with an OH group of the polysaccharide to form an ether function. The formyl group can react with an OH or NH₂ group of the polysaccharide to form a hemiacetal or hemiaminal function. The hydroxyl group can react with a COOH group of the polysaccharide to form an ester function. The sulfhydryl group can react with a COOH group of the polysaccharide to form a thioester function.

The hydrazino (—NH—NH₂) group can react with a CHO group of the polysaccharide to form a hydrazone function. The acylhydrazino group can react with a CHO group of the polysaccharide to form a carbonyl hydrazone ═NNHC(O)— group. The aminoxy group can react with a CHO group of the polysaccharide to form an oxime =NO-function. The carbodiimide group can react with a COOH group of the polysaccharide to give a CO—NR^{a}—CO—NH function, and an acid anhydride residue can react with an OH or NH₂ group of the polysaccharide to form an ester or amide function.

Preferably, the functional groups Z are identical and represent an epoxide or vinyl group, more preferably epoxide.

According to another advantageous embodiment, the functional groups Z are identical and chosen from the amino, vinyl, formyl, and carbodiimide groups, are preferably amino groups.

In particular, the crosslinking agent is chosen from hexamethylene diisocyanate, 4,4′-diphenylmethylene diisocyanate, 4-arm PEG20K-isocyanate, spermine (or 1,12-diamino-5,9-diazadodecane), spermidine (or 1,8-diamino-5-azaoctane), cadaverine (or 1,5-diaminopentane), putrescine (1,4-diaminobutane), poly(ethylene glycol) diamine, ethylenediamine, 1,4-butanediol diglycidyl ether (BDDE), 1,2,7,8-diepoxy-octane, poly(ethylene glycol) diglycidyl ether (PEGDGE), 1,2-bis(2,3-epoxypropoxy)ethane (EGDGE), 1,3-bis(3-glycidyloxypropyl)tetramethyldisiloxane, poly(dimethylsiloxane) terminated at each end by a diglycidyl ether (CAS number: 130167-23-6), poly(ethylene glycol) diacid, disuccinimidyl suberate, bis(sulfosuccinimidyl)suberate, sebacoyl chloride, 1,4-butane diisothiocyanate, divinyl sulfone (DVS), glutaraldehyde, polyethylene glycol, 1,5-pentanedithiol, adipic acid dihydrazide, bis-aminooxy-poly(ethylene glycol), diethylenetriaminepentaacetic acid dianhydride, and the mixtures thereof.

When the functional groups Z are epoxide groups, the crosslinking agent is preferably chosen from 1,4-butanediol diglycidyl ether (BDDE), 1,2,7,8-diepoxy-octane, poly(ethylene glycol) diglycidyl ether (PEGDGE), 1,2-bis(2,3-epoxypropoxy)ethane (EGDGE), 1,3-bis(3-glycidyloxypropyl)tetramethyldisiloxane, poly(dimethylsiloxane) terminated at each end by a diglycidyl ether (CAS number: 130167-23-6), hydroxyapatite beads modified to carry epoxy groups and the mixtures thereof.

More preferably, the crosslinking agent is chosen from 1,4-butanediol diglycidyl ether (BDDE), 1,2,7,8-diepoxy-octane, poly(ethylene glycol) diglycidyl ether (PEGDGE), 1,2-bis(2,3-epoxypropoxy)ethane (EGDGE), and the mixtures thereof.

More preferably, the crosslinking agent is 1,4-butanediol diglycidyl ether (BDDE).

When the functional groups Z are amino groups, the crosslinking agent is preferably a polyamine chosen from spermine (or 1,12-diamino-5,9-diazadodecane), spermidine (or 1,8-diamino-5-azaoctane), cadaverine (or 1,5-diaminopentane), putrescine (or 1,4-diaminobutane), their salts or a mixture thereof, more preferably the crosslinking agent is a polyamine chosen from spermine, spermidine, their salts and the mixtures thereof.

The crosslinking agent can be chosen from hydroxyapatite beads modified to carry epoxy groups, a compound of formula Chem. I as described below, and the mixtures thereof.

Preferably, the crosslinking agent is a compound of formula Chem. I:

Y—(Z)ₙ
wherein the functional groups Z, identical or different, are as defined above,
n is an integer greater than or equal to 2, in particular ranging from 2 to 8, preferably equal to 2,
Y is an, in particular aliphatic, polyvalent hydrocarbon group, having a valence of n and comprising 1 to 150 carbon atoms:
   wherein one or more (for example 1 to 150, or even 1 to 50 or even to 15 or even 1 or 2) CH₂ units are optionally replaced by one or more divalent units chosen from the arylenes; —O—; —S—; —S(O)—; —C(═O)—; —SO₂—; —N(R¹)—; and —[SiR²R³O]ₘ—SiR²R³— with:
R¹ representing a hydrogen atom, an aliphatic hydrocarbon group having 1 to 6 carbon atoms, or an aryl-(C1-C6)alkyl;
m is an integer between 1 and 20 and
R² and R³, identical or different, represent a hydrogen atom; a halogen atom; an —OR¹¹ group with R¹¹ representing a hydrogen atom, an aryl group or an aliphatic hydrocarbon group having 1 to 6 carbon atoms; an aryl; or an aliphatic hydrocarbon group having 1 to 6 carbon atoms optionally substituted by one or more groups chosen from a halogen atom, an aryl and a hydroxyl,
   said polyvalent group being non-substituted or substituted by one or more monovalent groups chosen from a halogen atom, a hydroxyl, an aryl-(C1-C6)alkyl, preferably non-substituted.

In particular, n is an integer ranging from 2 to 8, preferably n represents 2, 3 or 4, more preferably n is equal to 2.

Advantageously, R¹ represents a hydrogen atom or a (C1-C6)alkyl group.

In particular, R² and R³, identical or different, represent an aliphatic hydrocarbon group having 1 to 6 carbon atoms, more particularly a (C1-C6)alkyl group.

Preferably, in the definition of Y, the polyvalent hydrocarbon group can be a polyvalent aliphatic or aromatic hydrocarbon group, preferably aliphatic and in particular saturated, having a valence of n and having 1 to 150 carbon atoms, preferably 1 to 50 carbon atoms, more preferably 1 to 20 carbon atoms, yet more preferably 2 to 20 carbon atoms.

In particular, in the definition of Y, the polyvalent group hydrocarbon is a saturated, in particular linear, polyvalent aliphatic hydrocarbon group.

Preferably, Y is a polyvalent hydrocarbon group as described above, wherein one or more CH₂ units are optionally replaced by one or more divalent units chosen from —O—, —SO₂—, —[SiR²R³O]ₘ—SiR²R³— and —NH—, with R², R³ and m as described above.

In particular, Y is a polyvalent hydrocarbon group as described above, preferably aliphatic unsaturated, and in particular linear, branched, or star-shaped, and optionally wherein:
at least two CH₂ units replaced by —O—, in particular between 1 and 50 CH₂ units, more particularly between 1 and 15 CH₂ units, or
at least one, preferably one or two, CH₂ unit is replaced by an —NH— unit, or
at least one, preferably one, CH₂ unit is replaced by an —SO₂— unit, or
at least two, preferably two, CH₂ units are replaced by —O— and at least one, preferably one, CH₂ unit is replaced by an —[SiR²R³O]ₘ—SiR²R³— unit with R², R³ and m as described above.

More particularly, when one or more CH₂ units are replaced by —O—, the one or more units replaced are such as Y comprises one or more —CH₂—CH₂—O— units.

In particular, Y comprises 1 to 50 —CH₂—CH₂—O— units, advantageously 2 to 25 —CH₂—CH₂—O— units, more advantageously 2 to 15 —CH₂—CH₂—O— units. Y may comprise only —CH₂—CH₂—O— units.

More preferably, Y is a preferably linear alkyl group comprising 1 to 150, in particular 1to 50, in particular 1 to 20, for example 1 to 12, in particular 1 to 6 carbon atoms, wherein optionally one or more CH₂ units are replaced by one or more divalent units chosen from —O— and —NH—, more particularly between 1 and 50, in particular between 1 and 15, for example 1 or 2, divalent units chosen from —O— and —NH—.

According to a first embodiment, R² and R³, identical or different, represent an —OR¹¹ group with R¹¹ as described above. In particular, R¹¹ represents an aliphatic hydrocarbon group having 1 to 6 carbon atoms, more particularly a (C1-C6)alkyl group.

According to a second embodiment, R² and R³, identical or different, represent an aliphatic hydrocarbon group having 1 to 6 carbon atoms optionally substituted (preferably non-substituted) by one or more groups chosen from a halogen atom, an aryl or a hydroxyl, more preferably a non-substituted (C1-C6)alkyl groups such as a methyl or ethyl.

Advantageously, the crosslinking agent is a compound of following formula Chem. Ia:

Z¹—Y¹—Z²
wherein the Z¹ and Z² groups, identical or different, are chosen from the isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy and carbodiimide groups, and an acid anhydride residue, and Y¹ represents an, in particular aliphatic, divalent hydrocarbon chain, having 1 to 50 carbon atoms:
wherein one or more (for example 1 to 15 or even 1 or 2) CH₂ units are optionally replaced by one or more divalent units chosen from the arylenes, —O—, —S—, —S(O)—, —C(═O)—, —SO₂—, —N(R¹)— and —[SiR²R³O]ₘ—SiR²R³— with
R¹ representing a hydrogen atom, an aliphatic hydrocarbon group having 1 to 6 carbon atoms, or an aryl-(C1-C6)alkyl, m is an integer between 2 and 20, and
R² and R³, identical or different, represent a hydrogen atom, halogen atom; an —OR¹¹ group with R¹¹ representing a hydrogen atom, an aryl group or an aliphatic hydrocarbon group having 1 to 6 carbon atoms; an aryl; or an aliphatic hydrocarbon group having 1 to 6 carbon atoms optionally substituted by one or more groups chosen from a halogen atom, an aryl and a hydroxyl,
said chain being non-substituted or substituted by one or more monovalent groups chosen from a halogen atom, a hydroxyl, an aryl-(C1-C6)alkyl group.

The Z¹ and Z² groups have the same definition as the Z group defined above.

Y¹ have the same definition as Y defined above with a valence not being equal to 2. In particular Y¹ may comprise only —CH₂—CH₂—O— units, as previously defined.

Preferably, the crosslinking agent of formula Chem. I or Chem. Ia does not comprise —[SiR²R³O]ₘ—SiR²R³— units.

### Functionalisation Agent

The functionalisation agent enables a crosslinking of the polysaccharide by sol-gel reaction. Thus, the functionalisation agent typically comprises a single function capable of reacting with a functional group of the polysaccharide and comprises a silylated group capable of reacting with another silylated group via a sol-gel reaction so as to enable the crosslinking of the polysaccharide and to form a hydrogel.

The functionalisation agent is typically a molecule of formula Chem. II as shown below:

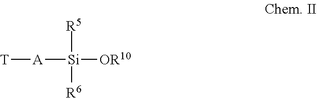

or a salt thereof,
wherein:
T represents an isocyanate, amino, epoxide, carboxyl, N-succinimidyloxycarbonyl, N-sulfosuccinimidyloxycarbonyl halogenocarbonyl, isothiocyanate, vinyl, formyl, hydroxyl, sulfhydryl, hydrazino, acylhydrazino, aminoxy, carbodiimide group, or an acid anhydride residue;
A represents a chemical bond or a spacer group;
R⁵ and R⁶, identical or different, represent a hydrogen atom; a halogen atom; an —OR⁴ group with R⁴ representing a hydrogen atom, an aryl group or an aliphatic hydrocarbon group having 1 to 6 carbon atoms; an aryl; or an aliphatic hydrocarbon group having 1 to 6 carbon atoms optionally substituted by one or more groups chosen from a halogen atom, an aryl and a hydroxyl;
R¹⁰ represents a hydrogen atom, an aryl group or an aliphatic hydrocarbon group having 1 to 6 carbon atoms.

Preferably, in formula Chem. II, T represents an isocyanate, sulfhydryl, amino, epoxide, vinyl, formyl, or carbodiimide group, more advantageously, T represents an epoxide or amino group, yet more advantageously T represents an epoxide group.

Preferably, in formula Chem. II, A represents a spacer group, more preferably a divalent aliphatic hydrocarbon chain, in particular linear or branched, and saturated, having 1 to 12 carbon atoms:
wherein one or more (in particular 1, 2, 3 or 4) divalent units are optionally intercalated between two carbon atoms of said chain, chosen from the arylenes, —O—, —S—, —S(O)—, —C(═O)—, —SO₂— and —N(R⁹)— with R⁹ representing a hydrogen atom, an aliphatic hydrocarbon group having 1 to 6 carbon atoms, or an aryl-(C1-C6)alkyl group,
said chain being non-substituted or substituted by one or more monovalent groups chosen from a halogen atom, a hydroxyl, an aryl-(C1-C6)alkyl group.

Advantageously, A is an, in particular linear or branched and saturated, aliphatic divalent hydrocarbon chain, in which one or more —O— divalent units, more advantageously 1 to 4 —O— divalent units, yet more advantageously one divalent—O— unit, are optionally intercalated between two carbon atoms of said chain.

Preferably, A is a (C1-C12)alkylene chain, wherein one or more divalent —O— units, more preferably 1 to 4 divalent —O— units, yet more preferably one divalent —O— unit, are optionally intercalated between two carbon atoms of said chain.

In particular, A represents a divalent-(C1-C6)alkylene —O—(C1-C6)alkylene- chain, in particular —(C1-C4)alkylene-O—(C1-C4)alkylene-, more particularly a divalent —CH₂—O—(CH₂)3— chain, the CH₂ group being bonded to T and the (CH₂)₃ group being bonded to Si in the molecule of formula Chem. II.

Advantageously, the spacer group will also make it possible to avoid steric hindrance between the silylated group and the T group of the molecule of formula Chem. II, ensuring a stable bond between these two groups.

Preferably, in formula Chem. II, R⁵ and R⁶, identical or different, representing an —OR⁴ group with R⁴ representing a hydrogen atom, an aryl group or an aliphatic hydrocarbon group having 1 to 6 carbon atoms; or an aliphatic hydrocarbon group having 1 to 6 carbon atoms optionally substituted by one or more groups chosen from a halogen atom, an aryl and a hydroxyl group.

In particular, R⁵ and R⁶, identical or different, represent an —OR⁴ group with R⁴ representing an (C1-C6)alkyl group; or a (C1-C6)alkyl group.

Advantageously, R⁵ and R⁶, identical or different, represent an —OR⁴ group with R⁴ representing a hydrogen atom, an aryl group or an aliphatic hydrocarbon group having 1 to 6 carbon atoms, preferably with R⁴ representing an aliphatic hydrocarbon group having 1 to 6 carbon atoms, such as a (C1-C6)alkyl group.

Preferably, in formula Chem. II, R¹⁰ represents a hydrogen atom or an aliphatic hydrocarbon group having 1 to 6 carbon atoms such as a (C1-C6)alkyl group, more advantageously R¹⁰ represents an aliphatic hydrocarbon group having 1 to 6 carbon atoms such as a (C1-C6)alkyl group.

Preferably, the molecule of formula Chem. II is such that:
T is as defined above and advantageously represents an amino or epoxide group, preferably an epoxide group;
A is a divalent 13 (C1-C6)alkylene —O—((C1-C6)alkylene-chain, in particular —(C1-C4)alkylene-O—(C1-C4)alkylene-, such as —CH₂—O—(CH₂)₃—, the CH₂ group preferably being bonded to T and the (CH₂)₃ group being bonded to Si in the molecule of formula Chem. II;
R⁵ and R⁶, identical or different, are each an —OR⁴ group with R⁴ representing a (C1-C6)alkyl group, preferably a methyl or an ethyl group; or a (C1-C6)alkyl group, preferably a methyl or a ethyl group; and
R¹⁰ is a (C1-C6)alkyl the group, preferably methyl or ethyl; the R⁵, R⁶ and OR¹⁰ groups being able to be identical.

In particular, the molecule of formula Chem. II is chosen from the (3-aminopropyl)triethoxysilane (APTES), (3-glycidyloxypropyl)trimethoxysilane (GPTMS), 3-glycidoxypropyldimethoxymethylsilane, 3-glycidoxypropyldimethylethoxysilane, (3-glycidyloxypropyl)ethoxydimethoxysilane, (3-glycidyloxypropyl)triethoxysilane, diethoxy(3-glycidyloxypropyl)methylsilane, and the mixtures thereof; preferably from (3-glycidyloxypropyl)trimethoxysilane (GPTMS), (3-glycidyloxypropyl)ethoxydimethoxysilane, (3-glycidyloxypropyl)triethoxysilane, diethoxy(3-glycidyloxypropyl)methylsilane, and the mixtures thereof; yet more preferably (3-aminopropyl)triethoxysilane (APTES), (3-glycidyloxypropyl)trimethoxysilane (GPTMS) and the mixtures thereof.

### Preparation of the Reaction Medium (Step c))

Step c) of the method according to the invention comprises preparing a reaction medium comprising a solvent, the one or more polysaccharides and the one or more crosslinking agents and/or functionalisation agents.

The solvent is typically water or a mixture comprising water and an organic solvent (typically a mixture comprising at least 90% by weight water, or at least 95% or at least 99% by weight water relative to the total weight of the solvent).

For example, an organic solvent such as an alcohol, in particular ethanol, or DMSO, can be used to solubilise the crosslinking agent, for example when it involves poly(dimethylsiloxane) terminated at each end by a diglycidyl ether (CAS number: 130167-23-6), before its addition to form the reaction medium.

The reaction medium can further comprise salts, pH adjusters, for example a Bronsted base, more preferably a hydroxide salt, such as sodium or potassium hydroxide, additional components as described above and the mixtures thereof.

The addition of a Bronsted base may be particularly necessary when the functional groups Z of the crosslinking agent, such as Z¹ or Z², represent an epoxide group or a vinyl group. In these cases, the crosslinking generally takes place at a pH greater than or equal to 10, more advantageously greater than or equal to 12, which requires the addition of a Bronsted base to the reaction medium, typically at a concentration between 0.10 M and 0.30 M.

The total quantity of crosslinking agent in the reaction medium typically varies from 0.001 to 0.15 mole per 1 mole of polysaccharide repetition unit, preferably from 0.001 to 0.08 mole per 1 mole of polysaccharide repetition unit, more preferably from 0.001 to 0.05 mole per 1 mole of polysaccharide repetition unit, yet more preferably from 0.001 to 0.03 mole per 1 mole of polysaccharide repetition unit.

In particular, the total quantity of crosslinking agent in the reaction medium varies from 0.001 to 0.02 mole per 1 mole of polysaccharide repetition unit, preferably from 0.001 to 0.015 mole, more preferably from 0.001 to 0.01 mole per 1 mole of polysaccharide repetition unit, yet more preferably from 0.001 to 0.008 mole or from 0.001 to 0.005 mole or from 0.001 to 0.004 moles per 1 mole of polysaccharide repetition unit. When the polysaccharide is a glycosaminoglycan such as a hyaluronic acid, the repetition unit is a disaccharide unit.

The total quantity of functionalisation agent in the reaction medium typically varies from 0.01 to 0.50, preferably from 0.05 to 0.45, in particular from 0.10 to 0.25 mole per 1 mole of polysaccharide repetition unit. For example, the total quantity of formula Chem. II or a salt thereof in the reaction medium typically varies from 0.01 to 0.50, preferably from 0.05 to 0.45, in particular from 0.10 to 0.25 moles per 1 mole of polysaccharide repetition unit.

Typically, the higher the weight average molar mass Mw of the polysaccharide, the lower will be the functionalisation ratio with a view to obtaining a hydrogel having equivalent mechanical properties, in particular equivalent viscoelastic properties (in particular elastic modulus G′, stress at the intersection of G′ and G″ and/or phase angle δ). In other words, the higher the weight average molar mass Mw of the polysaccharide, the lower will be the molar quantity of functionalisation agent in the reaction medium, for example in the molecule of formula Chem. II.

The functionalisation of the polysaccharide is typically carried out in an aqueous reaction medium.

The mass concentration of polysaccharide or polysaccharide salt in the reaction medium advantageously varies from 50 to 300 mg/g of solvent, preferably from 100 to 250 mg/g, preferably from 100 to 200 mg/g.

Step c) of the method according to the invention typically comprises a homogenisation

step of the reaction medium. The homogenisation is generally carried out by three-dimensional stirring, stirring with a mixer, stirring with blades or stirring with a spatula.

Step c) is typically carried out at a temperature ranging from 4 to 35° C., preferably ranging from 10° C. to 30° C., preferably ranging from 15° C. to 25° C.

Step c) is typically carried out at atmospheric pressure.

In particular, step c) is carried out at atmospheric pressure and at a temperature ranging from 4 to 35° C., preferably ranging from 10° C. to 30° C., preferably ranging from 15° C. to 25° C.

The duration of the preparation step of the reaction medium does not typically exceed 5 hours. It generally varies from 15 minutes to 4 hours, preferably from 30 minutes to 2 hours.

The reaction medium is typically prepared from polysaccharide or polysaccharides in dried form. When the reaction medium is prepared from polysaccharide or polysaccharides in hydrated form, the non-crosslinked aqueous gel or the aqueous polysaccharide solution used for the preparation of the reaction medium does not typically comprise sodium hydroxide. Thus, the maximum time of contact of the polysaccharide with sodium hydroxide before starting step d), whether the polysaccharide is provided in dried or hydrated form, is generally 5 hours, for example from 15 minutes to 4 hours or from 30 minutes to 2 hours.

### Crosslinking the Polysaccharide (Step d))

Step d) of the method according to the invention consists of crosslinking the one or more polysaccharides.

The crosslinking can be carried out by reaction of the one or more polysaccharides with the crosslinking agent (step d1) or by sol-gel reaction of the polysaccharide functionalised by reaction of the polysaccharide with the functionalisation agent (step d2).

The crosslinking of the polysaccharide according to step d) (d1) or d2)) is carried out at pressure P.

The crosslinking of the polysaccharide according to d1) or d2) is carried out under conditions not allowing sublimation of water, at a temperature T greater than the temperature of the eutectic point of the reaction medium as measured at pressure P and less than the temperature of the freezing point of the reaction medium as measured at pressure P.

The pressure P is advantageously equal to atmospheric pressure or less than atmospheric pressure.

The crosslinking of the polysaccharide according to step d) (d1) or d2)) can be carried out under vacuum, in particular at a pressure P less than atmospheric pressure, preferably at a pressure P between 0.7.105 and 0.9.105 Pa (between 0.7 and 0.9 bar), preferably between 0.7.105 and 0.8.105 Pa (between 0.7 and 0.8 bar).

The temperature of the freezing point of the reaction medium designates the temperature at which, at the considered pressure P, the mixture of the components of the reaction medium, on the macroscopic scale, solidify, in other words become non-fluid. Below the freezing point, the mixture is in a frozen state which is characterised by the coexistence of components in solid and liquid form. The freezing state is maintained down to the temperature of the eutectic point of the reaction medium.

The temperature of the eutectic point of the reaction medium designates the temperature below which, at the considered pressure P, the mixture of the components of the reaction medium passes from a frozen state (coexistence of liquid and solid phases) to a completely solid state, in other words a state in which all the components of the mixture are in solid form.

The freezing point and the eutectic point of a mixture depend on the pressure to which the mixture is subjected, therefore the freezing point and the eutectic point are measured at pressure P.

The freezing point and the eutectic point can be determined by differential scanning calorimetry. This method makes it possible to determine the phase transitions. For this purpose, the product to be studied is gradually cooled until its phase transitions are observed.

Using a device in which gaseous exchanges are not possible, enables the crosslinking of the polysaccharide according to d1) or d2) to be carried out under conditions not allowing sublimation of water. The method may comprise a step prior to step d), of degassing in order to remove air bubbles from the mixture once it is placed in the device.

In certain embodiments, the crosslinking is thus carried out in a sealed flexible hermetic container (for example a flexible hermetic bag), from which the air has been evacuated.

The crosslinking is then said to be carried out under vacuum or in a flexible hermetic container under vacuum. Typically, when the crosslinking of the polysaccharide according to d1) or d2) is carried out under vacuum, the reaction medium is placed or prepared in the flexible hermetic container. At least 90% by volume, preferably at least 95% by volume, yet more preferably at least 99% by volume, of the air contained in the container comprising the reaction medium is then removed, for example by means of a vacuum sealer. Before the step of evacuating and sealing, the method may comprise a degassing step in order to remove air bubbles from the mixture. A flexible hermetic bag suitable for the method according to the invention is described, for example, in patent EP 2 429 486 B1.

In certain embodiments, the crosslinking is thus carried out in a closed rigid hermetic container. In the case of a rigid hermetic container, sublimation of water is prevented by saturating any free volume left by the reaction medium in the container with water vapour or by ensuring that the reaction medium occupies at least 95% by volume, or at least 98% by volume, or at least 99% by volume of the volume of the container.

The hermetic container is advantageously placed at a temperature ranging from −35° C. to −10° C., at atmospheric pressure, preferably at a temperature of approximately −20° C., at atmospheric pressure.

Step d) is typically carried out, at least partially (in other words in part or entirely), under conditions not allowing sublimation of water and at temperature T, at pressure P.

Advantageously according to the invention, during step d), the variation in mass of the hydrogel is zero or represents at most 1% by mass, preferably at most 0.5% by mass, more preferably at most 0.1% by mass, yet more preferably at most 0.05% by mass relative to the total mass of the hydrogel resulting from step d). The variation in the mass of the hydrogel can be checked by weighing or by gravimetric analysis. A zero or almost zero variation in mass of the hydrogel can confirm that there has been no sublimation of water.

Step d) is typically performed for a duration of at least 1 hour, preferably at least 3 hours, preferably at least 72 hours, preferably at most 27 weeks. Preferably, step d) is performed for a duration ranging from 2 to 25 weeks, preferably ranging from 2 to 20 weeks or 2 to 17 weeks, yet more preferably from 3 to 8 weeks or 4 to 7 weeks under conditions not allowing sublimation of water and at temperature T, at pressure P.

### Step d1

The crosslinking of the polysaccharide by reaction of the polysaccharide with the crosslinking agent has mainly taken place during step d), but it can nevertheless start from step c).

Step d1) enables crosslinking of the polysaccharide chains with one another. The functional groups of the crosslinking agent react with functional groups present on the polysaccharides, in particular the functional groups naturally present on the polysaccharides, so as to bond the polysaccharide chains to one another and to crosslink them by forming intermolecular bonds. The crosslinking agent can also react with the functional groups present on a same polysaccharide molecule so as to form intramolecular bonds. Notably, the functional groups of the crosslinking agent react with —OH or —COOH, or even —CHO groups naturally present on the polysaccharides, such as hyaluronic acid. Crosslinked polysaccharides comprising at least one crosslinking bond between two polysaccharide chains, said crosslinking bond being the residue of the crosslinking agent, are thus obtained.

In particular, following step d), the cross-linked polysaccharides comprise at least one crosslinking bond between two polysaccharide chains, said crosslinking bond comprising more particularly the polyvalent group Y as described above, preferably, the divalent group Y¹ as described above. Certain functional groups Z (such as Z¹ and Z²) of the crosslinking agent may however not react with a polysaccharide chain. In particular, when the crosslinking agent includes two functional groups Z¹ and Z², one of the Z¹ functional groups can react with a polysaccharide while the other Z² functional group does not react with any polysaccharide. A dangling bond is then formed.

The crosslinking can be carried out in the presence of a plurality of crosslinking agents.

When the crosslinking is carried out in the presence of a plurality of crosslinking agents, the crosslinking agents can be added simultaneously or at separate times to the reaction medium. Step d1) can thus comprise repeated crosslinking steps.

The crosslinking is then typically carried out in the presence of a total quantity of crosslinking agents (or their salts) ranging from 0.001 to 0.15 mole per 1 mole of polysaccharide repetition unit, preferably from 0.001 to 0.08 mole per 1 mole of polysaccharide repetition unit, more preferably from 0.001 to 0.05 mole per 1 mole of polysaccharide repetition unit, yet more preferably from 0.001 to 0.03 mole of cross-linking agents per 1 mole of polysaccharide repetition unit. In particular, the cross-linking is carried out in the presence of a total quantity of crosslinking agents (or their salts) ranging from 0.001 to 0.02 mole per 1 mole of polysaccharide repetition unit, preferably from 0.001 to 0.015 mole, more preferably from 0.001 to 0.01 mole per 1 mole of polysaccharide repetition unit, yet more preferably from 0.001 to 0.008 mole or from 0.001 to 0.005 mole or from 0.001 to 0.004 mole per 1 mole of polysaccharide repetition unit. The crosslinking conditions, in particular the percentage of crosslinking agent, duration and temperatures as well as the weight averaged molar masses (Mw) of the polysaccharide, used are interdependent.

More particularly, the higher the crosslinking reaction temperature, the shorter will be the reaction time in order to obtain the same degree of modification of the polysaccharide by the crosslinking agent.

The lower the percentage of crosslinking agent, the longer the duration of the reaction must be in order to obtain equivalent mechanical properties of the resultant gel. In other words, the lower the molar percentage of crosslinking agent, the fewer will be the reactive functions in the reaction medium and the lower will be the probability that 2 groups meet and react together, thus the longer the duration of the reaction must be in order to enable the functions to react with one another and to form crosslinking bonds, and thus to obtain a gel with desirable properties.

Conversely, too large a quantity of crosslinking agent and too long a crosslinking time will lead to a brittle gel that is too hard, and therefore not of interest for a filling and/or cosmetic application.

The higher the weight average molar mass (Mw) of the polysaccharide, the lower the degree of modification necessary for obtaining gels with given mechanical properties.

For a given molar percentage of crosslinking agent, the lower the weight average molar mass (Mw) of the polysaccharide, the longer the duration of the reaction in order to obtain gels with given mechanical properties.

When the functional groups Z of the crosslinking agent are amino groups, the crosslinking reaction with the polysaccharide is advantageously carried out in the presence of at least one activator, and where appropriate combined with at least one coupling auxiliary.

In this respect, the activator can be selected from the water-soluble carbodiimides, such as 1-ethyl-3-(3-dimethylaminopropyl)carbodiimide (EDC), 1-ethyl-3-[3-(trimethylamino)propyl]carbodiimide hydrochloride (ETC), the 1-cyclohexyl-3-(2-morphilinoethyl)carbodiimide (CMC), their salts and the mixtures thereof, is preferably EDC.

With respect to the coupling auxiliary, when it is present, it can be selected from N-hydroxy succinimide (NHS), N-hydroxybenzotriazole (HOBt), 3,4-dihydro-3-hydroxy-4-oxo-1,2,3-benzotriazole (HOOBt), 1-hydroxy-7-azabenzotriazole (Hat) and N-hydroxysylfosuccinimide (sulfo NHS), and the mixtures thereof, is preferably HOBt.

In certain embodiments, during step d1), the reaction medium obtained at the end of step c) is placed, for a duration of at least 1 hour, preferably at least 3 hours, preferably at least 72 hours, preferably at most 27 weeks, under conditions not allowing sublimation of water (e.g. a flexible hermetic container under vacuum or rigid hermetic container, the free volume of which is saturated with water vapour or in which the reaction medium occupies at least 95% of the volume) and at a temperature T greater than the temperature of the eutectic point of the reaction medium (in other words of the mixture comprising the one or more polysaccharides, the one or more crosslinking agents, the solvent and any salts, pH regulators and additional components) as measured at pressure P and less than the temperature of the freezing point of the reaction medium as measured at pressure P.

In certain embodiments, during step d1) the reaction medium obtained at the end of step c) is placed, for a duration ranging from 2 to 27 weeks, preferably ranging from 2 to 20 or 2 to 17 weeks, yet more preferably from 3 to 8 weeks or 4 to 7 weeks, under conditions not allowing sublimation of water (e.g. a flexible hermetic container under vacuum or rigid hermetic container, the free volume of which is saturated with water vapour or in which the reaction medium occupies at least 95% of the volume) and at a temperature T greater than the temperature of the eutectic point of the reaction medium (in other words of the mixture comprising the one or more polysaccharides, the one or more crosslinking agents, the solvent and any salts, pH regulators and additional components) as measured at pressure P and less than the temperature of the freezing point of the reaction medium as measured at pressure P. The pressure P is advantageously less than or equal to atmospheric pressure.

The crosslinking of the polysaccharide according to step d1) can be carried out under vacuum, in particular at a pressure P less than atmospheric pressure, preferably at a pressure P between 0.7.105 and 0.9.105 Pa (between 0.7 and 0.9 bar), preferably between 0.7.105 and 0.8.105 Pa (between 0.7 and 0.8 bar).

During step d1), the reaction medium obtained at the end of step c) is advantageously placed in a flexible hermetic container. After an optional degassing step, the flexible hermetic container is placed under vacuum and advantageously sealed. This flexible hermetic container under vacuum is left at atmospheric pressure at a temperature greater than or equal to −55° C. and less than or equal to −5° C., preferably at a temperature ranging from −35° C. to −10° C., yet more preferably, at a temperature of approximately −20° C.

### Step d2

Step d2) consists of crosslinking by sol-gel reaction of the polysaccharide functionalised by a reaction of the polysaccharide with the functionalisation agent.

In other words, step d2) therefore comprises a functionalisation of the polysaccharide by reaction of the polysaccharide with the functionalisation agent and a sol-gel reaction of the functionalised polysaccharide. The functionalisation of the polysaccharide and the sol-gel reaction can be sequential or at least partially concomitant.

### Functionalisation of the Polysaccharide

The polysaccharide is typically functionalised with at least one molecule of formula Chem. II as described above in such a way as to become a carrier of Si—OR groups which will be able to react together and lead to a crosslinked polysaccharide. Since the molecule of formula Chem. II comprises a single reactive function with regard to the polysaccharide and enables crosslinking only via a sol-gel reaction, it does not have the toxicity of conventional cross-linking agents: the molecule of formula Chem. II cannot directly crosslink with biological molecules (proteins, DNA, etc.). More specifically, the functional group T of the molecule of formula Chem. II reacts with a functional group present on the polysaccharides so as to functionalise the polysaccharide chains.

Notably, the functional group T of the molecule Chem. II thus reacts with an —OH or —COOH group, or even a CHO function, present on the polysaccharides, such as hyaluronic acid. Functionalised polysaccharides are thus obtained, comprising dangling bonds on a polysaccharide chain, said dangling bonds comprising a -A-Si(R⁵)(R⁶)OR¹⁰ group, the -A-Si(R⁵)(R⁶)OR¹⁰ group coming from the molecule of formula Chem. II being able to give the hydrogel biological properties.

The solvent is typically water or a mixture comprising water and an organic solvent (for example an alcohol, in particular ethanol, or DMSO; typically a mixture comprising at least 90% by weight water, or at least 95% or at least 99% by weight water relative to the total weight of the solvent).

The reaction medium typically comprises 0.01 to 0.50, preferably 0.05 to 0.45, in particular 0.10 to 0.25 mole of the molecule of formula Chem. II or a salt thereof, per 1 mole of polysaccharide repetition unit.

The mass concentration of polysaccharide of the reaction medium is advantageously between 50 and 300 mg/g of solvent, preferably between 100 and 200 mg/g.

In certain embodiments, in particular when T is an epoxide, the functionalisation is carried out at a pH greater than or equal to 9, or greater than or equal to 10, more advantageously greater than or equal to 12, and in particular at a pH less than 14, for example less than or equal to 13.5. For this purpose, the reaction medium preferably comprises a Bronsted base, more preferably a hydroxide, yet more preferably sodium or potassium hydroxide. Advantageously, the reaction medium comprises sodium or potassium hydroxide at a concentration between 0.10 M and 0.30 M.

In certain embodiments, in particular when T is an amino group, the functionalisation is carried out at a pH less than 7, more advantageously greater than or equal to 4.5 and less than 7 or less than or equal to 6.5. For this purpose, the reaction medium preferably comprises a Bronsted acid, more preferably hydrochloric acid, sulfuric acid, or acetic acid.

In certain embodiments, the functionalisation of the polysaccharide is carried out at atmospheric pressure and at a temperature between 4° C. and 60° C., more preferably between 10° C. and 50° C. In these embodiments, the duration of the functionalisation reaction can vary from 1 hour to 2 weeks, more particularly from 3 hours to 1 week, yet more particularly from 3 hours to 96 hours, for example from 3 hours to 80 hours, in particular from 3 hours to 75 hours.

In certain embodiments, in particular when the functionalisation and crosslinking of the polysaccharide are concomitant or partially concomitant, the functionalisation of the polysaccharide can be at least partially carried out under conditions not allowing sublimation of water and at a temperature T greater than the temperature of the eutectic point of the reaction medium as measured at pressure P and less than the temperature of the freezing point of the reaction medium as measured at pressure P. Here, the pressure P is advantageously atmospheric pressure.

The higher the functionalisation temperature, the shorter will be the functionalisation time in order to obtain the same degree of modification by functionalisation.

### Sol-Gel Reaction

The “sol-gel reaction” consists of forming Si—O—Si bonds from Si—OR groups, with R representing a hydrogen atom, an aryl group or an aliphatic hydrocarbon group having 1 to 6 carbon atoms. This reaction proceeds as follows:
(i) if R is not a hydrogen atom, a hydrolysis step of at least some of the Si—OR groups in order to give Si—OH groups; then
(ii) a step of condensing Si—OH groups two-by-two or a Si—OH group with a Si—OR group in order to form Si—O—Si bonds.

The functionalised polysaccharide is crosslinked by sol-gel reaction in order to give a hydrogel.

This step enables the polysaccharide chains to be cross-linked with one another when they are functionalised with molecules of formula Chem. II. More specifically, during this step, at least some of the Si—OR¹⁰ groups and optionally at least some of the SiOR⁴groups will react two-by-two, optionally after hydrolysis of these groups, to form Si—O—Si bonds. This implies that two molecules of formula Chem. II grafted on the polysaccharide chains will react together via their Si—OR¹⁰ (or SiOR⁴, as applicable) end groups and covalently bond via the formation of a Si—O—Si bond thus enabling bonding together of the polysaccharide chains and their crosslinking.

In this way, crosslinked polysaccharides are obtained, comprising crosslinking bonds between two polysaccharide chains, said crosslinking bonds comprising a divalent —Si—O—Si— group.

The step d2) is carried out at least partially (in other words in part or entirely), under conditions not allowing sublimation of water and at a pressure P and a temperature T greater than the temperature of the eutectic point of the reaction medium as measured at pressure P and less than the temperature of the freezing point of the reaction medium as measured at pressure P.

The pressure P is advantageously less than or equal to atmospheric pressure.

The crosslinking of the polysaccharide according to step d2) can be carried out under vacuum, in particular at a pressure P less than atmospheric pressure, preferably at a pressure P between 0.7.105 and 0.9.105 Pa (between 0.7 and 0.9 bar), preferably between 0.7.105 and 0.8.105 Pa (between 0.7 and 0.8 bar).

The pressure P is advantageously atmospheric pressure.

In certain embodiments, the sol-gel reaction is carried out in a hermetically sealed container which can be rigid or flexible, as previously described.

Thus, during step d2), the reaction medium obtained at the end of step c) can advantageously be placed in a flexible hermetic container. After an optional degassing step, the flexible hermetic container is placed under vacuum and advantageously sealed.

This flexible hermetic container under vacuum is left at atmospheric pressure at a temperature greater than or equal to −55° C. and less than or equal to −5° C., preferably at a temperature ranging from −35° C. to −10° C., yet more preferably, at a temperature of approximately −20° C. The pressure P is then less than atmospheric pressure, preferably at a pressure P between 0.7.105 and 0.9.105 Pa (between 0.7 and 0.9 bar), preferably between 0.7.105 and 0.8.105 Pa (between 0.7 and 0.8 bar).

The hermetic container is advantageously a rigid hermetic container.

In the case of a rigid hermetic container, sublimation of water is prevented by saturating any free volume left by the reaction medium in the container with water vapour or by ensuring that the reaction medium occupies at least 95% by volume, or at least 98% by volume, or at least 99% by volume of the volume of the container. The pressure P is advantageously atmospheric pressure.

Step d2) is typically performed for a duration of at least 1 hour, preferably at least 3 hours, preferably at least 72 hours, preferably at most 27 weeks.

The duration of maintaining these conditions depends on the pH of the reaction medium.

Thus, when the pH of the reaction medium is greater than or equal to 9, or greater than or equal to 10, and less than 14, the temperature T is maintained for a duration t ranging from at least 1 hour, preferably of at least 3 hours, preferably of at least 72 hours, preferably at most 27 weeks. Advantageously, the temperature T is maintained for a duration t ranging from 2 to 27 weeks, in particular from 2 to 20 weeks or from 2 to 17 weeks, for example from 3 to 8 weeks or from 4 to 7 weeks. When the pH of the reaction medium is greater than or equal to 6.8 and less than or equal to 7.8, the temperature T is maintained for a duration t ranging from 1 to 48 hours, preferably greater than or equal to 6 hours and less than or equal to 36 hours, in particular greater than or equal to 7 hours and less than or equal to 36 hours.

It should be understood that the reaction conditions (pH, T, P, conditions not allowing sublimation of water) disclosed above can correspond to conditions applied throughout the entire duration of the crosslinking step (step d2)) or may correspond to conditions applied for only a part of the duration of step d2). In other words, the duration of crosslinking step d2) can be greater than the durations t indicated above, the reaction conditions (pH, P or T) applied in the additional time then being different from those disclosed above.

The sol-gel reaction typically takes place in an aqueous reaction medium.

The mass concentration of polysaccharide of the reaction medium is advantageously between 50 and 300 mg/g of sol-gel reaction medium, preferably between 100 and 250 mg/g, preferably between 100 and 200 mg/g.

Preferably, the pressure P is atmospheric pressure and the temperature T is greater than or equal to −55° C. and less than or equal to −5° C., preferably it ranges from −35° C. to −10° C., in particular from −30° C. to −10° C. or from −25° C. to −15° C. Yet more preferably, temperature T is approximately −20° C. at atmospheric pressure.

Advantageously, the reaction medium is placed and maintained at temperature T at atmospheric pressure by contact of the container comprising the reaction medium with air or a liquid L at temperature T. The liquid L may be, in particular, ethylene glycol, glycerol or an azeotropic mixture of these with water. The liquid L will be chosen as a function of the desired temperature T, so as to be liquid at this temperature T at atmospheric pressure. More advantageously, the reaction medium is left at temperature T at atmospheric pressure by contact of the container comprising the reaction medium with air at temperature T.

Typically, the lower the temperature T, the longer the duration t in order to obtain hydrogels having equivalent mechanical properties. Indeed, the lower the temperature T, the less kinetic is the sol-gel reaction.

Similarly, the lower the functionalisation ratio, the longer the duration t in order to obtain hydrogels having equivalent mechanical properties.

In other words, the lower the molar quantity of molecule of formula Chem. II or a salt thereof per 1 mole of polysaccharide repetition unit, the fewer are the Si—OH functions in the reaction medium and the lower will be the probability that 2 groups meet and react together, thus the longer the duration t must be in order to enable the Si—OH functions to react with one another and to form crosslinking bonds, and thus to obtain a gel with desirable properties.

For a same molar quantity of molecule of formula Chem. II or a salt thereof per 1 mole of polysaccharide repetition unit, the lower the weight average molar mass Mw of the polysaccharide, the longer the duration t in order to obtain hydrogels having equivalent mechanical properties.

When the crosslinking by sol-gel reaction at temperature T is carried out in a reaction medium at a pH greater than or equal to 9, or greater than or equal to 10, and less than 14, the reaction medium preferably comprises a Bronsted base, more preferably a hydroxide, yet more preferably sodium or potassium hydroxide. Advantageously, the reaction medium comprises sodium or potassium hydroxide at a concentration between 0.10 M and 0.30 M.

Preferably, at the end of the duration t (crosslinking carried out under conditions not allowing sublimation of water and the temperature T, at pressure P), the pH of the reaction medium is adjusted to a physiological pH, preferably to a pH of approximately 6.8 to 7.8. It should be understood that at the end of the duration t, the reaction medium is brought back to ambient temperature at atmospheric pressure.

When the crosslinking by sol-gel reaction at temperature T, at pressure P, under conditions not allowing sublimation of water (e.g. flexible hermetic container under vacuum or rigid hermetic container, the free volume of which is saturated with water vapour, or in which the reaction medium occupies at least 95% of the volume) is carried out in a reaction medium at physiological pH (pH greater than or equal to 6.8 and less than or equal to 7.8) and when the functionalisation is carried out in a base medium (pH greater than or equal to 9, or greater than or equal to 10, and less than 14), the pH of the reaction medium will be brought to a physiological pH before the temperature is brought to temperature T under conditions not allowing sublimation of water. In this case, the method will advantageously comprise a neutralisation step of the gel in order to reach this physiological pH, before the reaction medium is brought to temperature T under conditions not allowing sublimation of water. For this purpose, a Bronsted acid is preferably added to the reaction medium, preferably an aqueous solution of hydrochloric acid, an aqueous solution of sulfuric acid or an aqueous solution of acetic acid.

When the crosslinking by sol-gel reaction at temperature T, at pressure P, under conditions not allowing sublimation of water (e.g. flexible hermetic container under vacuum or rigid hermetic container, the free volume of which is saturated with water vapour, or in which the reaction medium occupies at least 95% of the volume) is carried out in a reaction medium at physiological pH (pH greater than or equal to 6.8 and less than or equal to 7.8) and when the functionalisation is carried out at a pH less than 7, for example greater than or equal to 4.5, and less than 7 or less than or equal to 6.5, the pH of the reaction medium will be brought to a physiological pH before the temperature is brought to temperature T under conditions not allowing sublimation of water.

During step d2), the pressure P is advantageously atmospheric pressure.

Very generally, the method of the present invention comprises concomitantly or partially concomitantly carrying out steps c) and d2). A concomitant carrying out of steps c) and d2) makes it possible to shorten the duration of the method for preparing of the hydrogel and to simplify it.

The method of the present invention therefore comprises steps a) to d2) as described above and is characterised in that steps c) and d2) are concomitant or partially concomitant. The method then comprises:
functionalisation and crosslinking by sol-gel reaction carried out in a reaction medium at a pH greater than or equal to 9, or greater than or equal to 10, and less than 14, under conditions not allowing sublimation of water (e.g. rigid hermetic container, the free volume of which is saturated with water vapour or in which the reaction medium occupies at least 95% of the volume), at pressure P and at temperature T greater than the temperature of the eutectic point of the reaction medium as measured at pressure P and less than the temperature of the freezing point of the reaction medium as measured at pressure P, for a duration t ranging from 2 weeks to 27 weeks, in particular from 2 to 20 weeks or from 2 to 17 weeks, for example from 3 to 8 weeks or 4 to 7 weeks, or
a functionalisation and a crosslinking by sol-gel reaction carried out in a reaction medium at a pH greater than or equal to 6.8 and less than or equal to 7.8 under conditions not allowing sublimation of water (e.g. rigid hermetic container, the free volume of which is saturated with water vapour or in which the reaction medium occupies at least 95% of the volume), at pressure P and at temperature T greater than the temperature of the eutectic point of the reaction medium as measured at pressure P and less than the temperature of the freezing point of the reaction medium as measured at pressure P, for a duration t between 1 hour and 48 hours, preferably greater than or equal to 6 hours and less than or equal to 36 hours, in particular greater than or equal to 7 hours and less than or equal to 36 hours.

The pressure P is advantageously atmospheric pressure.

It should be understood from the above that steps c) and d) are at least partially (in part or in full) carried out under conditions disclosed above (T, P, pH, conditions not allowing sublimation of water, t).

In certain embodiments, the functionalisation of the polysaccharide and the crosslinking of the functionalised polysaccharide are then carried out as follows:
1) preparing a reaction medium comprising the one or more polysaccharides, the one or more molecules of formula Chem. II and a solvent, the pH of the reaction medium being greater than or equal to 9, or greater than or equal to 10, and less than 14;
2) optionally placing the reaction medium at a temperature ranging from 4° C. to 60° C., preferably from 10° C. to 50° C., at atmospheric pressure, typically for a duration ranging from 1 hour to 2 weeks, more particularly from 3 hours to 1 week, for example from 3 hours to 80 hours, in particular from 3 hours to 75 hours;
3) optionally adjusting the pH of the reaction medium to a pH greater than or equal to 6.8 and less than or equal to 7.8;
4) placing the reaction medium:
   under conditions not allowing sublimation of water (e.g. rigid hermetic container, the free volume of which is saturated with water vapour or in which the reaction medium occupies at least 95% of the volume), at atmospheric pressure, and at a temperature T greater than the temperature of the eutectic point of the reaction medium as measured at atmospheric pressure and less than the temperature of the freezing point of the reaction medium as measured at atmospheric pressure, for a duration t ranging from 2 weeks to 27 weeks, when the pH of the reaction medium is greater than or equal to 9, or greater than or equal to 10, and less than 14, in particular from 2 to 20 weeks or from 2to 17 weeks, for example from 3 to 8 weeks or 4 to 7 weeks, or
   under conditions not allowing sublimation of water (e.g. rigid hermetic container, the free volume of which is saturated with water vapour or in which the reaction medium occupies at least 95% of the volume), at atmospheric pressure, and at a temperature T greater than the temperature of the eutectic point of the reaction medium as measured at atmospheric pressure and less than the temperature of the freezing point of the reaction medium as measured at atmospheric pressure, for a duration t between 1 hour and 48 hours, preferably greater than or equal to 6 hours and less than or equal to 36 hours, in particular greater than or equal to 7 hours and less than or equal to 36 hours, when the pH of the reaction medium is greater than or equal to 6.8 and less than or equal to 7.8.

In other words, the method of the present invention can then be defined in the following manner:
a) providing at least one polysaccharide;
b) providing at least one molecule of formula Chem. II as described above;
c) functionalisation of the polysaccharide with at least one molecule of formula Chem. II as described above;
d) crosslinking by sol-gel reaction of the functionalised polysaccharide in order to give a hydrogel; wherein the functionalisation and the crosslinking of the functionalised polysaccharide are carried out in the following manner:
1) preparing a reaction medium comprising the one or more polysaccharides, the one or more molecules of formula Chem. II and a solvent, the pH of the reaction medium being greater than or equal to 9, or greater than or equal to 10, and less than 14;
2) optionally placing the reaction medium at atmospheric pressure and at a temperature ranging from 4° C. to 60° C., preferably from 10° C. to 50° C., typically for a duration ranging from 1 hour to 2 weeks, more particularly from 3 hours to 1 week, for example from 3 hours to 80 hours, in particular from 3 hours to 75 hours;
3) optionally adjusting the pH of the reaction medium to a pH greater than or equal to 6.8 and less than or equal to 7.8;
4) placing the reaction medium:
   under conditions not allowing sublimation of water (e.g. rigid hermetic container, the free volume of which is saturated with water vapour or in which the reaction medium occupies at least 95% of the volume), at atmospheric pressure and at a temperature T greater than the temperature of the eutectic point of the reaction medium as measured at atmospheric pressure and less than the temperature of the freezing point of the reaction medium as measured at atmospheric pressure, for a duration t ranging from 2 weeks to 27 weeks, in particular from 2 to 20 weeks or from 2 to 17 weeks, for example from 3 to 8 weeks or 4 to 7 weeks, when the pH of the reaction medium is greater than or equal to 9, or greater than or equal to 10, and less than 14, or
   under conditions not allowing sublimation of water (e.g. rigid hermetic container, the free volume of which is saturated with water vapour or in which the reaction medium occupies at least 95% of the volume), at atmospheric pressure and at a temperature T greater than the temperature of the eutectic point of the reaction medium as measured at atmospheric pressure and less than the temperature of the freezing point of the reaction medium as measured at atmospheric pressure, for a duration t between 1 hour and 48 hours, preferably greater than or equal to 6 hours and less than or equal to 36 hours, in particular greater than or equal to 7 hours and less than or equal to 36 hours, when the pH of the reaction medium is greater than or equal to 6.8 and less than 7.8.

In certain embodiments, the functionalisation and the crosslinking are carried out in a reaction medium for which the pH is greater than or equal to 9, or greater than or equal to 10, and less than 14. For this purpose, the reaction medium preferably comprises a Bronsted base, more preferably a hydroxide, yet more preferably sodium or potassium hydroxide. Advantageously, the reaction medium comprises sodium or potassium hydroxide at a concentration between 0.10 M and 0.30 M.

In these embodiments, according to a first variant, the reaction medium prepared in step 1) can be placed at atmospheric pressure and at a temperature ranging from 4° C. to 60° C., preferably from 10° C. to 50° C., typically for a duration ranging from 1 hour to 2 weeks, more particularly from 3 hours to 1 week, for example from 3 hours to 80 hours, in particular from 3 hours to 75 hours (step 2) before being placed at temperature T, at atmospheric pressure, under conditions not allowing sublimation of water for a duration t (step 4). During step 2), the polysaccharide will be functionalised and some of the Si—OR¹⁰ groups and optionally some of the Si—OR⁴ groups will condense with each other (pre-condensation), with more Si—OR¹⁰ groups and optionally some Si—OR⁴ groups condensing during step 4) (advance condensation).

In these embodiments, according to a second variant, the reaction medium prepared in step 1) can be placed directly at the end of step 1) at temperature T, at atmospheric pressure under conditions not allowing sublimation of water for a duration t (step 4).

At the end of time t (first and second variant), the temperature of the reaction medium is typically returned to ambient temperature. The pH of the reaction medium is then preferably brought to a physiological pH (approximately 6.8 to 7.8). For this purpose, a Bronsted acid is preferably added to the reaction medium, preferably an aqueous solution of hydrochloric acid, an aqueous solution of sulfuric acid or an aqueous solution of acetic acid.

In certain embodiments, the crosslinking is carried out partially in a reaction medium, for which the pH is greater than or equal to 6.8 and less than 7.8.

In these embodiments, a reaction medium having a pH greater than or equal to 9, or greater than or equal to 10, and less than 14 and comprising the one or more polysaccharides, the one or more molecules of formula Chem. II and a solvent is prepared (step 1)).

The reaction medium is placed at a temperature ranging from 4° C. to 60° C., preferably from 10° C. to 50° C., typically for a duration ranging from 1 hour to 2 weeks, more particularly from 3 hours to 1 week, for example from 3 hours to 80 hours, in particular from 3 hours to 75 hours (step 2). Then, the pH of the reaction medium is brought to a physiological pH (step 3)) before the temperature is brought to T at atmospheric pressure under conditions not allowing sublimation of water. In this case, the method will advantageously comprise a neutralisation step of the gel in order to reach this physiological pH, before the reaction medium is brought at atmospheric pressure to temperature T under conditions not allowing sublimation of water. For this purpose, a Bronsted acid is preferably added to the reaction medium, preferably an aqueous solution of hydrochloric acid, an aqueous solution of sulfuric acid or an aqueous solution of acetic acid. The reaction medium is then placed at temperature T at atmospheric pressure under conditions not allowing sublimation of water for a duration t between 1 hour and 48 hours (step 4).

Preferably, the method of the present invention comprises only a single step of placing the reaction medium at temperature T at atmospheric pressure under conditions not allowing sublimation of water.

### Optional Steps

The method according to the invention can comprise one or more additional steps, such as steps of adding one or more additional components for purification, sterilisation, sieving, swelling and/or packaging.

They can be carried out:
before the reaction medium is at temperature T during step d),
or after step d), in other words after the reaction medium has been maintained at temperature T.

The method according to the invention can comprise a step of adding at least one additional component. The additional component can be chosen from lubricants; cosmetic active ingredients such as antioxidants, co-enzymes, amino acids, vitamins, minerals and nucleic acids; therapeutic active ingredients such as anaesthetics, antibiotics, antifungals and adrenaline and its derivatives, and the mixtures thereof. The additional components can be as described above.

The method according to the invention can comprise at least one purification step.

The purification can be carried out by dialysis.

The method according to the invention can comprise a step of sterilising the hydrogel.

The sterilisation is preferably carried out by heat. The sterilisation is generally carried out by increasing the temperature of the sterilisation medium up to a so-called “plateau temperature”, which is maintained for a determined so-called “plateau duration”. The sterilisation is preferably carried out at a plateau temperature ranging from 121° C. to 135° C., preferably for a plateau duration ranging from 1 minute to 20 minutes with F0≥15. The sterilising value F0 corresponds to the time necessary, in minutes, at 121° C., to inactivate 90% of the population of microorganisms present in the product to be sterilised. Alternatively, the sterilisation can be carried out, in particular, by radiation with gamma rays, UV or by means of ethylene oxide.

The method can comprise a step of sieving the hydrogel, more particularly with a sieve having a porosity between 50 and 2000 μm. This sieving step makes it possible to obtain a more homogeneous hydrogel with the most constant possible extrusion force, i.e. the most regular possible extrusion force. A person skilled in the art knows to select a sieve with suitable pore size depending on the mechanical properties of the hydrogel undergoing preparation.

The method can comprise a step of swelling the hydrogel. During the step of swelling the hydrogel, the concentration of polysaccharide of the hydrogel is adjusted. In particular, a solvent is added, for example water, a phosphate buffer, water for an injectable preparation. More particularly, the added solvent has a pH that is approximately the physiological pH. The concentration of polysaccharide obtained following the swelling step advantageously varies from 1 mg/g of gel to 50 mg/g of hydrogel, more advantageously from 5 mg/g to 35 mg/g of hydrogel, yet more advantageously from 10 mg/g to 30 mg/g of hydrogel gel.

The method can comprise a step of packaging the hydrogel.

Where applicable, the step of adding one or more additional components preferably takes place after the purification step.

Where applicable, the step of addition of one or more additional components preferably takes place before the sterilisation step.

In particular, the step of adding one or more additional components can also comprise adding at least one therapeutic activity ingredient, or at least one cosmetic active ingredient, or the mixture thereof. When at least one therapeutic active ingredient and/or at least one cosmetic active ingredient is added, the step of adding one or more additional components preferably takes place after step d).

Where applicable, the purification step preferably takes place after the step of adding one or more additional components.

Where applicable, the purification step preferably takes place before the sterilisation step.

Where applicable, the purification step preferably takes place before the sieving step.

The sterilisation step is preferably carried out after steps a) to d) and the optional additional steps. In particular, the hydrogel is sterilised after having been packaged in its injection device and the packaging of the hydrogel takes place following all the steps of the method and before sterilisation.

When the polysaccharide is crosslinked according to step d2), the method according to the invention can also comprise an additional step of crosslinking the polysaccharide with a conventional crosslinking agent, and more particularly crosslinking of the polysaccharide provided in step a) or of the crosslinked polysaccharide obtained following step d2), in the presence of at least one crosslinking agent or of a salt thereof, said crosslinking agent comprising at least two functional groups Z as described above.

The method according to the invention can also comprise a step of adding a molecule of the following formula Chem. III:
R⁷O—[R¹²R¹³SiO]ₚ—R⁸
or a salt thereof
wherein:
   p is an integer from 1 to 20;
R¹² and R¹³, identical or different, represent a hydrogen atom; a halogen atom; an —OR¹⁴ group with R¹⁴ representing a hydrogen atom, an aryl group or an aliphatic hydrocarbon group having 1 to 6 carbon atoms; an aryl; or an aliphatic hydrocarbon group having 1 to 6 carbon atoms optionally substituted by one or more groups chosen from a halogen atom, an aryl and a hydroxyl; and
   R⁷ and R⁸, identical or different, represent a hydrogen atom, an aryl group or an aliphatic hydrocarbon group having 1 to 6 carbon atoms.

According to an embodiment, the step of adding a molecule of formula Chem. III is carried out before the temperature of the reaction medium is at temperature T under conditions not allowing sublimation of water during step d), in particular before, during or after step c).

According to a variant, this step can be carried out after step d), in other words after the reaction medium has been maintained at temperature T under conditions not allowing sublimation of water.

When step c) and step d) are concomitant, this step can be carried out before these steps c) and d), in particular between step a) and step c). Preferably, R¹² and R¹³, identical or different, represent an —OR¹⁴ group with R¹⁴

representing a hydrogen atom, an aryl group or an aliphatic hydrocarbon group having 1 to 6 carbon atoms; an aryl; an aliphatic hydrocarbon group having 1 to 6 carbon atoms optionally substituted by one or more groups chosen from a halogen atom, an aryl and a hydroxyl.

In particular, R₁₂ and R₁₃, identical or different, represent an —OR₁₄ group with R₁₄ representing a hydrogen atom, an aliphatic hydrocarbon group having 1 to 6 carbon atoms, preferably a (C1-C6)alkyl group; or an aliphatic hydrocarbon group having 1 to 6 carbon atoms, preferably a (C1-C6)alkyl group.

Advantageously, R⁷ and R⁸, identical or different, represent a hydrogen atom, or an aliphatic hydrocarbon group having 1 to 6 carbon atoms, preferably a (C1-C6)alkyl group.

This molecule of formula Chem. III comprises Si—OR (Si—OR⁷, Si—OR⁸ groups and optionally Si—OR¹⁴) capable of reacting with the Si—OR (Si—OR¹⁰ groups and optionally Si—OR⁴) of the molecule of formula Chem. II. Thus, during the sol-gel reaction enabling the formation of Si—O—Si bonds, a molecule of formula Chem. III can bond to two molecules of formula Chem. II grafted on polysaccharide chains so as to form crosslinking bonds resulting from the coupling of a molecule of formula Chem. III with two molecules of formula Chem. II.

For example, the molecule of formula Chem. III is orthosilicic acid, tetraethyl orthosilicate (TEOS), polydimethylsiloxane (PDMS), oligomerised TEOS/orthosilicic acid, or methyl silanetriol (preferably used in the form of its sodium salt called sodium methyl siliconate—NAMS).

Advantageously, the step of adding a molecule of formula Chem. III takes place at a pH greater than or equal to 9, in particular greater than or equal to 10, more advantageously greater than or equal to 12, and in particular less than 14, for example less than or equal to 13.5, in particular when the molecule of formula Chem. III is sodium methyl siliconate (NAMS).

For this purpose, the reaction medium preferably comprises a Bronsted base, more preferably a hydroxide, yet more preferably sodium or potassium hydroxide. In particular, the reaction medium comprises a Bronsted base, more preferably a hydroxide, yet more preferably sodium or potassium hydroxide at a concentration between 0.10 M and 0.30 M.

### Hydrogel

Another object of the present invention is a hydrogel which can be obtained by the method of the present invention. Such a hydrogel may also be designated by the term “cryogel” in the present description.

The liquid medium of the hydrogel is preferably an aqueous medium chosen from an aqueous solution or a mixture of aqueous solutions, preferably chosen from water for injectable preparation, phosphate saline buffer or a mixture of the two, more preferably phosphate saline buffer in the context of therapeutic, cosmetic and aesthetic applications according to the invention.

The hydrogel comprises one or more polysaccharides as defined above.

The polysaccharide is crosslinked. The molar crosslinking ratio of the polysaccharide is greater than 0 and less than or equal to 15%, preferably less than or equal to 8%, preferably less than or equal to 5%, preferably less than or equal to 3%, preferably less than or equal to 2%, preferably less than or equal to 1.5%, preferably less than or equal to 1%, in particular between 0.1% and 0.8%, more preferably less than or equal to 0.5%, in particular between 0.1% and 0.4% (number of moles of crosslinking agent(s) per 100 moles of repetition unit of the one or more polysaccharides).

The one or more crosslinking agents are as defined above.

The crosslinking agent is preferably a crosslinking agent for which the functional groups are epoxide groups. Thus, the crosslinking agent is preferably 1,4-butanediol diglycidyl ether (BDDE), 1,2,7,8-diepoxy-octane, poly(ethylene glycol) diglycidyl ether (PEGDGE), 1,2-bis(2,3-epoxypropoxy)ethane (EGDGE), and the mixtures thereof.

The hydrogel of the present invention advantageously has a stickiness property.

The hydrogel of the present invention advantageously has a stretchiness property.

The hydrogel is preferably an injectable hydrogel.

It is preferably sterile, in particular sterilised by heat at a plateau temperature of 121° C. to 135° C., preferably for a plateau duration ranging from 1 minute to 20 minutes with F0≥15.

This hydrogel is preferably homogeneous.

This hydrogel may also comprise an additional component chosen from lubricants; cosmetic active ingredients such as antioxidants, co-enzymes, amino acids, vitamins, minerals, and nucleic acids; and the mixtures thereof, as described below.

This hydrogel may also comprise at least one therapeutic active ingredient advantageously chosen from anaesthetics, antibiotics, antifungals, adrenaline and its derivatives, and the mixtures thereof, as described below.

The polysaccharide of this hydrogel is preferably as defined above, in the context of the description of step a) of the method according to the invention.

Preferably, a hydrogel according to the present invention, acceptable for the therapeutic and/or cosmetic applications targeted by the present invention, has a cross-over stress (or stress at the intersection of the G′ and G″ moduli) greater than or equal to 50 Pa, preferably between 50 and 5000 Pa and more preferably between 100 and 1000 Pa and an elastic modulus G′ greater than or equal to 20 Pa, preferably from 100 Pa to 2000 Pa, more preferably from 100 Pa to 1000 Pa.

Preferably, a hydrogel according to the present invention, acceptable for the therapeutic and/or cosmetic applications targeted by the present invention, has a cohesiveness of 1 N to 30 N. This cohesiveness is measured by mechanical compression using a rheometer. For this purpose, the gel is deposited on a Peltier plane with an initial gap of 2.60 mm; it is then compressed at constant speed of 100 um/s to 70% of the initial gap, at 25° C.; finally, the cohesiveness of the gel is measured at the end of the compression stroke. The more cohesive a gel is, i.e. the higher its value of cohesiveness, the more it is able to withstand stresses, such as those it may encounter after being administered to a subject.

### Compositions

Another object of the present invention is a composition comprising the hydrogel according to the present invention. It preferably involves a cosmetic or pharmaceutical composition. It may further comprise physiologically acceptable excipients.

The hydrogel according to the invention comprises a crosslinked polysaccharide, preferably hyaluronic acid. The composition may further comprise a non-crosslinked polysaccharide, preferably hyaluronic acid.

The non-crosslinked hyaluronic acid can be present in the composition as a lubricant.

The hydrogel according to the present invention can thus comprise 0.1 to 10% by weight, preferably 0.1 to 5% by weight, preferably 1 to 3% by weight polysaccharide, preferably hyaluronic acid, relative to the total weight of said composition, the polysaccharide such as hyaluronic acid, being present in crosslinked and optionally non-crosslinked form. In particular, the percentage of non-crosslinked polysaccharide, in particular hyaluronic acid, varies from 0 to 40% by weight, preferably from 1 to 40% by weight, more preferably from 5 to 30% by weight, relative to the total weight of polysaccharide, in particular hyaluronic acid, present in the composition.

The composition according to the present invention is preferably a sterile composition, in particular sterilised by heat at a plateau temperature between 121° C. and 135° C., preferably for a plateau duration between 1 minute and 20 minutes, with F0≥15. It is preferably an injectable composition. The composition according to the invention therefore preferably comprises a physiologically acceptable medium, preferably a physiologically acceptable aqueous medium.

The physiologically acceptable aqueous medium may comprise a solvent or a mixture of physiologically acceptable solvents and preferably comprises water, preferably the solvent is water.

The physiologically acceptable medium may also comprise isotonic agents such as oses, sodium chloride and the mixtures thereof.

The physiologically acceptable medium may further comprise at least one isotonic and physiologically acceptable saline solution.

Said balanced saline solution is preferably a phosphate buffered saline solution, and in particular a KH₂PO₄/K₂HPO₄ buffered saline solution.

The composition according to the invention may further comprise at least one additional compound chosen from lubricants; cosmetic active ingredients such as antioxidants, co-enzymes, amino acids, vitamins, minerals, and nucleic acids; and the mixtures thereof.

Preferably, the additional compound is water-soluble or modified to be soluble in an aqueous medium.

The composition according to the invention may also comprise at least one therapeutic active ingredient advantageously chosen from anaesthetics, antibiotics, antifungals, adrenaline and its derivatives, and the mixtures thereof, as described below. The therapeutic active ingredient is preferably water-soluble.

Examples of anaesthetics include ambucaine, amoxecaine, amyleine, aprindine, aptocaine, articaine, benzocaine, betoxycaine, bupivacaine, butacaine, butamben, butanilicaine, chlorobutanol, chloroprocaine, cinchocaine, clodacaine, cocaine, cryofluorane, cyclomethycaine, dexivacaine, diamocaine, diperodon, dyclonine, etidocaine, euprocine, febuverine, fomocaine, guafecainol, heptacaine, hexylcaine, hydroxyprocaine, hydroxytetracaine, isobutamben, leucinocaine, levobupivacaine, levoxadrol, lidamidine, lidocaine, lotucaine, menglytate, mepivacaine, meprylcaine, myrtecaine, octacaine, octodrine, oxetacaine, oxybuprocaine, parethoxycaine, paridocaine, phenacaine, piperocaine, piridocaine, polidocanol, pramocaine, prilocaine, procaine, propanocaine, propipocaine, propoxycaine, proxymetacaine, pyrrocaine, quatacaine, quinisocaine, risocaine, rodocaine, ropivacaine, tetracaine, tolycaine, trimecaine or one of the salts thereof, in particular a hydrochloride thereof, and a mixture of these. Advantageously, the anaesthetic is chosen between lidocaine or one of the salts thereof, in particular a hydrochloride, and mepivacaine or one of the salts thereof, in particular a hydrochloride, or mixtures of these.

Examples of antioxidants include, in a non-limiting manner, glutathione, reduced glutathione, ellagic acid, spermine, resveratrol, retinol, L-carnitine, polyols, polyphenols, flavonols, theaflavins, catechins, caffeine, ubiquinol, ubiquinone, alpha-lipoic acid and their derivatives, and a mixture of these.

Examples of amino acids include, in a non-limiting manner, arginine (e.g., L-arginine), isoleucine (e.g., L-isoleucine), leucine (e.g., L-leucine), lysine (e.g., L-lysine or monohydrated L-lysine), glycine, valine (e.g., L-valine), threonine (e.g., L-threonine), proline (e.g., L-proline), methionine, histidine, phenylalanine, tryptophan, cysteine, their derivatives (e.g., N-acetyl derivatives such as N-acetyl-L-cysteine) and a mixture thereof.

Examples of vitamins and their salts include, in a non-limiting manner, vitamins E, A, C, B, especially vitamins B6, B8, B4, B5, B9, B7, B12, and preferably pyridoxine and its derivatives and/or salts, preferably pyridoxine hydrochloride.

Examples of minerals include, in a non-limiting manner, the salts of zinc (e.g., zinc acetate, in particular dehydrated zinc acetate), magnesium salts, calcium salts (e.g., hydroxyapatite, in particular in bead form), potassium salts, manganese salts, sodium salts, copper salts (e.g., copper sulphate, in particular pentahydrated copper sulphate), optionally in a hydrated form, and the mixtures thereof.

Nucleic acids include, in particular, adenosine, cytidine, guanosine, thymidine, cytodine, their derivatives and a mixture thereof.

Co-enzymes include the coenzyme Q10, CoA, NAD, NADP, and the mixtures thereof.

Adrenaline derivatives include noradrenaline.

The quantities of additional compounds depend of course on the nature of the compound in question, the desired effect, and the destination of the composition as described here.

### Applications

The hydrogel or the composition according to the invention can have therapeutic, cosmetic or aesthetic applications.

The present invention therefore also relates to a hydrogel or a composition according to the invention for its use in the filling and/or replacement of tissues, in particular soft tissues, in particular by injection of the hydrogel or the composition into the tissue.

The hydrogel or the composition may be intended for superficial application.

A superficial application refers to the administration of a composition in the upper layers of the skin, i.e. in or on the skin, for example by mesotherapy and, for example, for reducing superficial wrinkles and/or for improving the quality of the skin (such as its radiance, density or structure) and/or rejuvenating the skin.

The hydrogel or the composition may be intended for a deep application.

A “deep application” refers to the administration of a composition in the deepest layers of the skin and/or under the skin (above the periosteum) in order to increase the volume of the soft tissues, such as for filling deep wrinkles and/or partially atrophied regions of the face and/or body.

The hydrogel or the composition can be versatile, i.e., can be used for both deep and superficial applications.

Preferably, when the hydrogel or the composition according to the invention comprises at least one therapeutic active ingredient, the present invention relates to the hydrogel or a composition according to the invention for its use in the modified, delayed or prolonged release of therapeutic active ingredients.

In particular, the hydrogel or the composition according to the invention is used in oral healthcare and more particularly in the treatment of gingival recession, or for filling periodontal pockets. More particularly, the hydrogel or the composition according to the invention is used for treating defects in the gingival architecture which can occur with tooth loss, with ageing, with periodontal diseases and disorders, or after the insertion of tooth implants, crowns or bridges.

The hydrogel or the composition according to the invention can also be used in ophthalmology, more particularly for protecting the ocular structures during eye surgery, for example ophthalmic surgery of the anterior or posterior segment, the removal of cataracts optionally with implantation of an intraocular lens, cornea transplant surgery, glaucoma filtering surgery, or implantation of a secondary lens. In this case, the hydrogel or the composition according to the invention will be more particularly injected into the eye.

The hydrogel or the composition according to the invention can also be used in orthopaedics or rheumatology, for example by injection into the synovial cavity. The hydrogel or the composition according to the invention is then used as a viscosupplementation.

The hydrogel or the composition according to the invention can also be used in the treatment of lipodystrophy.

The hydrogel or the composition according to the invention can be used in aesthetic surgery, in particular for gynecoplastias and/or penoplastias.

The hydrogel or the composition according to the invention is administered, more particularly, by injection.

The hydrogel or the composition according to the invention can also be used for the modified, delayed or prolonged release of therapeutic active ingredients, in particular the therapeutic active ingredients as described above. The longer the hydrogel is left at temperature T° and pressure P in the presence of the one or more active ingredients, the greater will be the sol-gel reaction and the slower will be the release of the active ingredient. Thus, it is possible to adapt the duration and/or the intensity of release of the active ingredient to the requirement. This also applies to the modified, delayed or prolonged release of cosmetic active ingredients.

Another object of the present invention is the aesthetic use, and therefore non-therapeutic use, of a hydrogel or composition according to the invention for preventing and/or treating the alteration of the viscoelastic or biomechanical properties of the skin, and in particular to regenerate, hydrate, firm or restore the radiance of the skin, in particular by mesotherapy; to fill volume defects of the skin, and in particular to fill wrinkles, fine lines or scars (in particular hollow scars); in order to reduce the appearance of wrinkles and fine lines; or, when said hydrogel or said composition comprises at least one cosmetic active ingredient, for the modified, delayed or prolonged release of cosmetic active ingredients, in particular as defined above.

For example, an object of present invention is the aesthetic use of a hydrogel or a composition according to the invention for attenuating the nasolabial folds and bitterness folds; for increasing the volume of the cheekbones, the chin or lips; for restoring the volumes of the face, in particular the cheeks, temples, the oval of the face, and around the eye; or to regenerate, hydrate, firm or restore the radiance of the skin, in particular by mesotherapy.

In particular, the hydrogel or the composition according to the invention is an anti-ageing hydrogel or composition. The hydrogel or the composition according to the invention is administered, more particularly, by injection.

The present invention also relates to a method for cosmetic treatment, preferably anti-ageing treatment, of keratin materials, in particular the skin, comprising at least one step of administering a hydrogel or a composition according to the invention on or through said keratin materials, more particularly by injection.

The administration can be an injection, in particular an intra-epidermal and/or intradermal and/or subcutaneous injection. The administration by intra-epidermal and/or intradermal and/or subcutaneous injection according to the invention aims to inject a hydrogel or a composition of the invention in an epidermal, dermoepidermal and/or dermal region. The hydrogel or the composition according to the invention can also be administered by a supra-periosteum injection.

The hydrogel or composition according to the invention can be injected using any one of the methods known to a person skilled in the art. In particular, a hydrogel or a composition according to the invention can be administered by means of an injection device suitable for an intraepidermal and/or intradermal and/or subcutaneous and/or supraperiosteal injection. The injection device can be chosen, in particular, from a syringe, a set of microsyringes, a laser or hydraulic device, an injection gun, a needle-free injection device, or a microneedle roller.

The injection device may have any commonly used injection means suitable for an intraepidermal and/or intradermal and/or subcutaneous and/or supra-periosteum injection. Preferably, such a means can be a hypodermic needle or a cannula.

A needle or cannula according to the invention can have a diameter varying from 18 to 34 G, preferably between 25 and 32 G, and a length varying from 4 to 70 mm, and preferably from 4 to 25 mm. The needle or cannula is advantageously disposable.

Advantageously, the needle or cannula is combined with a syringe or any other device enabling said injectable hydrogel or composition to be delivered through the needle or cannula.

According to an alternative embodiment, a catheter can be inserted between the needle/cannula and the syringe. In known manner, the syringe can be manually activated by the practitioner or even by a syringe support such as guns.

Preferably, the injection device can be chosen from a syringe or a set of micro-syringes.

In an alternative embodiment, the injection device can be adapted to the mesotherapy technique.

Mesotherapy is a technique for treatment by intra-epidermal and/or intradermal and/or subcutaneous injection of a composition or hydrogel. The composition or the hydrogel is administered according to this technique by injection in the form of multiple small-sized droplets at the epidermis, the dermo-epidermal junction and/or the dermis in order, in particular, to produce a subcutaneous layer. The technique of mesotherapy is described, in particular, in the work entitled “Traité de mésothérapie” by Jacques LE COZ, published by Masson, 2004. Mesotherapy performed on the face is also called mesolift and also known by the term “mesoglow”.

The administration can be topical.

Preferably, it involves a topical application on the surface of the skin, more particularly on the epidermis, still more particularly on the facial epidermis.

The present invention also relates to an injection device as previously described comprising a hydrogel or a composition according to the invention.

The additional accessory biological effects of hydrogels according to the invention can be studied in vitro and/or in vivo and/or ex vivo; said in vivo tests can for example include administration tests, in small animals, of a composition according to the invention versus a comparative composition in order to follow the appearance of biological effects with, in particular, evaluation of the improvement in the quality of the skin in the animal, in particular the living human (e.g. its hydration and/or its elasticity) and, after sacrifice of the animal, histological sections in order to study any change in the protein expression at the site of administration (colouration).

These in vivo tests can generally include evaluating the quality of the skin in humans following the administration of a composition according to the invention vs. a comparative composition.

Said in vitro tests may include tests on dermal cells (such as fibroblasts) for cytotoxicity, viability, protein expression (ELISA) in particular for the expression of hyaluronic acid, elastin, fibrillin, aquaporin and/or various types of collagens and genetic expression (e.g., genes coding for hyaluronic acid, elastin, fibrillin, aquaporin and/or various types of collagens). The ex vivo tests, including the above mentioned tests, are carried out, for example, on explants of animal or human skin.

The present invention is illustrated by the non-limiting examples below.

### EXAMPLES

### 1.1 Equipment and Materials

1.5 MDa, 3 MDa and 4 MD non-crosslinked sodium hyaluronate
BDDE,
0.25 M NaOH
1 M HCl
Phosphate buffer
Three-dimensional stirrer
Rheometer DHR-2
Dynamometer and test bench
Paddle mill homogeniser
Polyethylene sterile bag

### 1.2 Methods

### 1.2.1 Measurement of Viscoelastic Properties

The viscoelastic properties of the hydrogels obtained have been measured using a rheometer (DHR-2) having a stainless-steel cone (1°-40 mm) with cone-plane geometry and a Peltier plane made of anodised aluminium (42 mm) (gap 24 μm). 0.5 g of sterilised hydrogel is deposited between the Peltier plane and said cone. Then a stress scan is performed at 1 Hz and 25° C. The elastic modulus G′, the viscous modulus G″ and the phase angle δ are reported for a stress of 5 Pa.

The stress at the intersection of G′ and G″, τ, is determined at the intersection of the curves of the G′ and G″ moduli and is expressed in Pascal.

### 1.2.2 Measurement of the Extrusion Force

The extrusion forces (in Newtons) of gels packaged in syringes were measured using a test bench equipped with the dynamometer at a constant speed of 12.5 mm/min, through a 27 G ½″ needle and at ambient temperature. The extrusion force results correspond to the average of the average extrusion forces on at least 2 samples.

### 1.3 Examples

Unless otherwise stated, the steps described below are carried out at ambient temperature (21° C.).

### 1.3.1 Example 1

Prototypes numbers 1 and 2 are prepared in the following way:
BDDE (crosslinking ratio given in table 1) and sodium hyaluronate (1.5 MDa, 120 mg/g) are dissolved in a 0.25 M solution of sodium hydroxide, in a sealed sterile bag.
The mixture is homogenised in the paddle mill for 3 cycles of 15 minutes at 210 rpm, the pH of the mixture is approximately 13,
The bag containing the mixture is degassed in order to remove air bubbles from the mixture and evacuated to 800 mbar and sealed using a sealer under vacuum (Solis Vac Premium Typ 574),
The bag containing the mixture and the vacuum is placed and held at −20° C., for a duration given in Table 1.
The bag containing the mixture under vacuum is left to thaw at ambient temperature for 3 hours before continuing the preparation,
A 1 M solution of HCl was added to the sterile bag until a pH of 7.3+0.5 was obtained,
In a container which may be different from the bag, the mixture is diluted to a concentration of 23 mg of hyaluronic acid per gram of mixture with the phosphate buffer solution PBS,
Sodium hyaluronate (4 MDa) is added as a lubricant then the mixture is homogenised.
The product thus obtained is sieved on the order of a micron and sterilised with the autoclave.

TABLE 1
crosslinking conditions

| Prototypes | Molar crosslinking ratio (TR) BDDE | Duration (days) |
|---|---|---|
| 1 | 0.8 | 62 |
| 2 | 0.4 | 60 |

After sterilisation, the prototypes are analysed, the elastic modulus G′, the phase angle δ and the stress at the intersection of G′ and G″, τ, are determined. The results are presented in Table 2 below.

TABLE 2
results.

| Prototypes | G′ (Pa) | δ (°) | τ (Pa) | F(N) |
|---|---|---|---|---|
| 1 | 704 ± 45 | 11.2 ± 0.5 | 194 ± 9 | 10.6 ± 0.5 |
| 2 | 292 ± 46 | 19.4 ± 2.0 | 350 ± 4 | 13.1 ± 0.2 |

The gels of prototypes 1 to 2 prepared according to the invention have good mechanical properties and have a phase angle δ less than or equal to 45°. The gels obtained in accordance with the method according to the invention 1 and 2 are transparent, with a smooth appearance and an absence of filaments.

### 1.3.2 Example 2

The prototypes A and B are prepared in the following way:

A common gel base was produced for the 2 tests (prototypes A and B) in the following way: 10 g of hyaluronic acid (3 MDa) were dissolved in 73 g of 0.25 M NaOH. The mixture was then homogenised in a paddle mill for 4 cycles of 15 minutes at 210 rpm. Next, 0.160 g of a BDDE solution diluted to ⅛th in the 0.25 M NaOH was added to the hyaluronic acid solution.

The HA-BDDE-NaOH mixture of the prototype A is placed in a sealed, tight sterile bag and not subjected to any other treatment, in particular not placed under vacuum.

The HA-BDDE-NaOH mixture of prototype B is placed in a sealed sterile bag. The mixture is degassed to remove the air bubbles from the mixture. Then, the bag comprising the degassed mixture is placed under vacuum at 800 mbar and sealed using a vacuum heat-sealer (Solis Vac Premium Typ 574). The bag is as described in patent EP2429486B1.

The bags comprising the mixtures of prototypes A and B are placed for 3 months (100 days) at −20° C. enabling the crosslinking of the mixtures. Photographs are taken after thawing (after 3 hours).

### Results

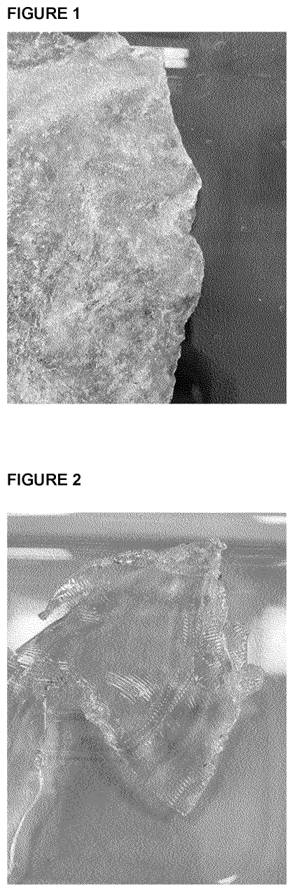

The results are presented in FIGS. 1 and 2.

The gel obtained in accordance with the method according to the invention (FIG. 2—prototype B) is transparent, it is characterised by a smooth appearance and an absence of filaments.

The gel obtained in FIG. 1 (prototype A) has inhomogeneous crosslinked regions, that are white and brittle.

1. A method for preparing a polysaccharide-based hydrogel comprising the following steps:
a) providing at least one polysaccharide;
b) providing at least one crosslinking agent and/or at least one functionalisation agent, the functionalisation agent enabling crosslinking of the polysaccharide by sol-gel reaction;
c) preparing a reaction medium comprising a solvent, the one or more polysaccharides and the one or more crosslinking agents and/or functionalisation agents;
d) crosslinking the polysaccharide:
d1) by reacting the polysaccharide with the one or more crosslinking agents; or
d2) by sol-gel reaction of the functionalised polysaccharide, the functionalised polysaccharide being obtained by reaction of the polysaccharide with the one or more functionalisation agents;
wherein the crosslinking of the polysaccharide according to d1) or d2) is carried out under conditions not allowing sublimation of water, at a pressure P and at a temperature T greater than the temperature of the eutectic point of the reaction medium as measured at pressure P and less than the temperature of the freezing point of the reaction medium as measured at pressure P.

2. The method according to claim 1, wherein the crosslinking agent comprises at least two functional groups Z, identical or different, chosen from the isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy or carbodiimide groups, and an acid anhydride residue.

3. The method according to claim 1, wherein the functionalisation agent is a molecule Chem. II having the following formula: 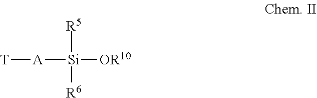
wherein:
T represents an isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy or carbodiimide group, or an acid anhydride residue;
A represents a chemical bond or a spacer group;
R5 and R6, identical or different, represent a hydrogen atom; a halogen atom; an —OR4 group with R4 representing a hydrogen atom, an aryl group or an aliphatic hydrocarbon group having 1 to 6 carbon atoms; an aryl; or an aliphatic hydrocarbon group having 1 to 6 carbon atoms optionally substituted by one or more groups chosen from a halogen atom, an aryl and a hydroxyl group;
R10 represents a hydrogen atom, an aryl group or an aliphatic hydrocarbon group having 1 to 6 carbon atoms.

4. The method according to claim 2, wherein the functional groups Z are identical and represent an epoxide or vinyl group, more preferably epoxide.

5. The method according to claim 1 wherein the crosslinking agent is selected from the group consisting of 1,4-butanediol diglycidyl ether (BDDE), 1,2,7,8-diepoxy-octane, poly(ethylene glycol) diglycidyl ether (PEGDGE), 1,2-bis(2,3-epoxypropoxy)ethane (EGDGE), 1,3-bis(3-glycidyloxypropyl)tetramethyldisiloxane, poly(dimethylsiloxane) terminated at each end by a diglycidyl ether (CAS number: 130167-23-6), hydroxyapatite beads modified to carry epoxy groups and the mixtures thereof.

6. The method according to claim 1, wherein the quantity of crosslinking agent varies from 0.001 to 0.15 mole per 1 mole of polysaccharide repetition unit.

7. The method according to claim 1, wherein the crosslinking of the polysaccharide according to d1) or d2) is carried out for a duration of at least 1 hour, under conditions not allowing sublimation of water at temperature T.

8. The method according to claim 1 wherein the crosslinking of the polysaccharide according to d1) or d2) is carried out for a duration ranging from 2 to 25 weeks, under conditions not allowing sublimation of water at temperature T.

9. The method according to claim 1 wherein the pressure P is less than or equal to atmospheric pressure.

10. The method according to claim 1 wherein step d) is carried out in a hermetically sealed container which can be flexible or rigid.

11. The method according to claim 10, wherein during step d) the hermetic container is placed at a temperature ranging from −35° C. to −10° C. at atmospheric pressure.

12. A hydrogel obtained by the method of claim 1.

13. A cosmetic or pharmaceutical composition comprising a hydrogel according to claim 12 and a physiologically acceptable excipient.

14. A cosmetic method for preventing and/or treating the alteration in viscoelastic or biomechanical properties of the skin; to fill wrinkles, fine lines and scars;
to reduce nasolabial folds and bitterness folds; to reduce the appearance of wrinkles and fine lines; or to stimulate, regenerate, hydrate, firm or restore the radiance of the skin, comprising administering to a subject the hydrogel according to claim 12 or a composition comprising the hydrogel according to claim 12 and a physiologically acceptable excipient.

15. The method according to claim 6 wherein the quantity of crosslinking agent varies from 0.001 to 0.08 mole per 1 mole of polysaccharide repetition unit.

16. The method according to claim 7, wherein the crosslinking of the polysaccharide according to d1) or d2) is carried out for a duration of at least 72 hours.

17. The method according to claim 7, wherein the crosslinking of the polysaccharide according to d1) or d2) is carried out for a duration of at most 27 weeks.

18. The method according to claim 8 wherein the crosslinking of the polysaccharide according to d1) or d2) is carried out for a duration ranging from 2 to 20 weeks.

19. The method according to claim 9 wherein the pressure P is between 0.7.105 Pa and 0.9.105 Pa or equal to atmospheric pressure.

20. The method according to claim 10 wherein step d) is carried out in a flexible hermetically sealed container.
